SEQUENCE LISTING (2 sequences)

SEQ ID NO: 1 - DNA, 2640 bp, Staphylococcus aureus
aacattctttgtggattgtttgacagtagacagtcatttgtatacgttaacgaattttgaaaaattgaatcattatttaatcataatattcattgcgcacgcgcaacacaaaaaggagacatgtcaatgacatatagaataaagaaatggcaaaaattatccaccattacgttattaatggctggtgtgattactttgaatggtggtgaattcagaagtgttgataaacatcaaatcgctgtggctgatacgaatgttcaaacgccagattatgaaaaattgaggaacacatggctggacgttaactatggttatgataagtatgatgagaataatccagatatgaagaagaagtttgatgctacagagaaagaggcgacgaatttactcaaggaaatgaaaactgaaagtggtaggaaatacttgtggtcaggagcggaaacccttgaaactaattcttctcatatgactcgtacctatcgtaatattgagaaaatcgcagaagcgatgaggaatcctaaaaccactttaaatactgacgaaaataagaagaaagtgaaagatgcccttgagtggctgcataaaaatgcatatggaaaagaaccagataaaaaagtaaaagaattaagtgagaattttactaaaacaactggcaagaataccaacttaaattggtgggattatgaaattggaacacctaaatcattaacaaatacgcttatattgctgaatgatcaattttcaaatgaagaaaagaaaaaattcactgcccctattaaaactttcgccccagatagtgacaaaatattatcttctgtaggaaaagctgaacttgctaaaggcggaaatctagtagacatttctaaggtgaaacttttagaatgtattatcgaagaagataaagatatgatgaaaaagtctatagattcatttaataaagtcttcacttacgttcaagattctgccactggtaaagaacgtaatggattttataaagatggctcttacattgatcatcaagacgttccatacactggtgcttatggcgttgtactcttagaggggatttctcaaatgatgccgatgataaaagaaacaccttttaatgataaaacccaaaatgatacaaccttaaagtcatggattgacgacggatttatgccactcatttataaaggtgaaatgatggatttatcacgaggtagagctatcagtcgtgaaaatgaaaccagtcactcagcatctgcaacagtaatgaaatcattgttgagattaagtgatgccatggatgattcaacaaaagctaagtataaaaagattgtcaaatcttcagtagagtcagattcaagttataaacaaaatgattatttaaattcatattcggacatagataaaatgaagtctttaatgacagataacagtatttctaaaaacggattaacacaacaacttaaaatatataatgacatggatcgtgtcacctatcataacaaagacttagactttgcatttggtttaagtatgacgtcgaaaaacgtagcacgctatgaaagtatcaacggagagaatttaaaaggttggcacactggtgctggaatgtcttatttatataacagcgatgtcaaacactatcatgataacttctgggtgacagccgatatgaaacgtttatcaggtacaacaactttagacaatgaaatattaaaagatacggatgataaaaagtcgagtaaaacttttgttggcggaacaaaagttgatgaccaacatgctagtatcggaatggattttgaaaatcaggacaaaactttaactgccaaaaaatcatatttcatattaaacgataaaattgtcttcttaggaactggcattaaaagtactgattcatcaaagaatccagttacaacgattgaaaatcgcaaagcgaatgggtatacgttatatacagacgataaacaaacaaccaattctgataatcaggaaaacaattcagtctttttagagtccacagataccaaaaagaacatcggttatcattttttaaacaaaccgaaaataactgtaaaaaaagaaagtcatactggtaagtggaaagaaataaataaaagtcaaaaggatacacaaaaaactgatgagtattatgaagtaactcaaaagcattctaattctgacaataaatatggatatgtgttgtatccaggcttatctaaagatgtctttaagacaaaaaaagatgaagtaactgtcgttaagcaagaagatgacttccacgttgtgaaagataatgaatcggtttgggctggtgttaattatagtaatagcactcaaacttttgacattaacaacactaaagtcgaagttaaagccaaaggtatgtttatacttaaaaagaaagatgataacacttatgaatgtagcttctataatcctgaatctacaaattccgcttcagatattgaatctaaaatttcaatgaccggttactctattacaaacaaaaatacgtcgacttctaatgaatccggcgtgcactttgaattaactaaataagcaagaaaatctcgacactataccatttttatagtgtcgagattttataagtatcgccatcattcagcttgtaatccgcagaccacatcc

SEQ ID NO: 2 - protein, 807 aa, Staphylococcus aureus
MTYRIKKWQKLSTITLLMAGVITLNGGEFRSVDKHQIAVADTNVQTPDYEKLRNTWLDVNYGYDKYDENNPDMKKKFDATEKEATNLLKEMKTESGRKYLWSGAETLETNSSHMTRTYRNIEKIAEAMRNPKTTLNTDENKKKVKDALEWLHKNAYGKEPDKKVKELSENFTKTTGKNTNLNWWDYEIGTPKSLTNTLILLNDQFSNEEKKKFTAPIKTFAPDSDKILSSVGKAELAKGGNLVDISKVKLLECIIEEDKDMMKKSIDSFNKVFTYVQDSATGKERNGFYKDGSYIDHQDVPYTGAYGVVLLEGISQMMPMIKETPFNDKTQNDTTLKSWIDDGFMPLIYKGEMMDLSRGRAISRENETSHSASATVMKSLLRLSDAMDDSTKAKYKKIVKSSVESDSSYKQNDYLNSYSDIDKMKSLMTDNSISKNGLTQQLKIYNDMDRVTYHNKDLDFAFGLSMTSKNVARYESINGENLKGWHTGAGMSYLYNSDVKHYHDNFWVTADMKRLSGTTTLDNEILKDTDDKKSSKTFVGGTKVDDQHASIGMDFENQDKTLTAKKSYFILNDKIVFLGTGIKSTDSSKNPVTTIENRKANGYTLYTDDKQTTNSDNQENNSVFLESTDTKKNIGYHFLNKPKITVKKESHTGKWKEINKSQKDTQKTDEYYEVTQKHSNSDNKYGYVLYPGLSKDVFKTKKDEVTVVKQEDDFHVVKDNESVWAGVNYSNSTQTFDINNTKVEVKAKGMFILKKKDDNTYECSFYNPESTNSASDIESKISMTGYSITNKNTSTSNESGVHFELTK

(19) United States
(12) Patent Application Publication
(10) Pub. No.: US 2022/0031777 A1
(43) Pub. Date: Feb. 3, 2022

(54) Methods of Treating Cancer

(71) Applicant: KaliVir Immunotherapeutics, Inc., Pittsburgh, PA (US)

(72) Inventor: Stephen H. THORNE, Pittsburgh, PA (US)

(21) Appl. No.: 17/367,788

(22) Filed: Jul. 6, 2021

Related U.S. Application Data
(63) Continuation of application No. PCT/US2020/012611, filed on Jan. 7, 2020.
(60) Provisional application No. 62/789,164, filed on Jan. 7, 2019.

(51) Int. Cl. A61K 35/76; A61K 35/74; A61P 35/00

(52) U.S. Cl. CPC A61K 35/76; A61P 35/00; A61K 35/74

(57) ABSTRACT

Disclosed herein are methods of treating cancer by administering a heterologous prime-boost regimen of oncolytic microorganisms that enhances or elicits an immune response to a tumor protein that is not coded for by the oncolytic microorganisms.

### CROSS-REFERENCE

This application is a bypass continuation of PCT/US2020/012611, filed Jan. 7, 2020, which claims the benefit of U.S. Provisional Application No. 62/789,164 filed Jan. 7, 2019, which is incorporated by reference herein in its entirety.

### BACKGROUND

Various immunotherapeutic approaches are being evaluated for the treatment of a number of cancers. However, there remains a need for immunotherapies and regimens capable of producing a robust immune response directed specifically to cancer cells.

### SUMMARY

The methods described herein feature, inter alia, methods of treating cancer by administering a heterologous prime-boost regimen of oncolytic microorganisms that enhances or elicits an immune response to a tumor protein that is not coded for by the oncolytic microorganisms.

One embodiment provides a method of treating a tumor in a subject, comprising: administering to the subject a first recombinant microorganism, wherein the first recombinant microorganism replicates in a tumor cell and does not replicate in a non-tumor cell or displays attenuated replication in a non-tumor cell, administering to the subject a second recombinant microorganism, wherein the second recombinant microorganism replicates in a tumor cell and does not replicate in a non-tumor cell or displays attenuated replication in a non-tumor cell, wherein the first recombinant microorganism and the second recombinant microorganism are different, and at least one of the following: a) enhancing or eliciting an immune response to a tumor protein that is not coded for or is not expressed by the first and second microorganisms, b) enhancing or eliciting an immune response to a protein expressed by a tumor associated cell that is not coded for or expressed by the first and second recombinant microorganisms. In some embodiments, the immune response is demonstrated by one or more of a decrease in the volume of the tumor in the subject, a decrease in the level of expression of one or more tumor proteins in the subject or a sample from the subject, a decrease in the number of tumor sites in the subject, a change in viral load in the subject or the sample from the subject, a change in population of immune cells in the subject or the sample from the subject, a change in expression levels of an immune cell marker in the subject or the sample from the subject, an enhancement of B-cell proliferation in the subject or the sample from the subject, an enhancement of CD4+ T cell proliferation in the subject or the sample from the subject, an enhancement of CD8+ T cells proliferation in the subject or the sample from the subject, an enhancement of cytokine production in the subject or the sample from the subject, an enhancement of antigen presenting cell proliferation in the subject or the sample from the subject, or any combinations thereof. In some embodiments, the immune response is demonstrated by the decrease in the level of expression of one or more tumor proteins, the change in population of immune cells, the change in expression levels of an immune cell marker, the enhancement of B-cell proliferation, the enhancement of CD4+ T cell proliferation, the enhancement of CD8+ T cells proliferation, the enhancement of cytokine production, the enhancement of antigen presenting cell proliferation, or any combinations thereof, in the subject or the sample from the subject, wherein the sample from the subject is a blood, tissue, urine, or saliva sample.

In some embodiments, the immune response can be detected at a time point at or after the administration of the first or second recombinant microorganism. In some embodiments, the first recombinant microorganism does not replicate in the non-tumor cell. In some embodiments, the first recombinant microorganism displays attenuated replication in the non-tumor cell. In some embodiments, the infectivity of the first recombinant microorganism is at least 70%, 75%, 80%, 85%, 90%, 95%, 96%, 97%, 98%, or 99% lower in the non-tumor cell relative to the tumor cell. In some embodiments, the replication efficiency of the first recombinant microorganism is at least 70%, 75%, 80%, 85%, 90%, 95%, 96%, 97%, 98%, or 99% lower in the non-tumor cell relative to the tumor cell. In some embodiments, the second recombinant microorganism does not replicate in the non-tumor cell. In some embodiments, the second recombinant microorganism displays attenuated replication in the non-tumor cell. In some embodiments, the infectivity of the second recombinant microorganism is at least 70%, 75%, 80%, 85%, 90%, 95%, 96%, 97%, 98%, or 99% lower in the non-tumor cell relative to the tumor cell. In some embodiments, the replication efficiency of the second recombinant microorganism is at least 70%, 75%, 80%, 85%, 90%, 95%, 96%, 97%, 98%, or 99% lower in the non-tumor cell relative to the tumor cell. In some embodiments, the first recombinant microorganism is a bacterium, virus, or parasite. In some embodiments, the second recombinant microorganism is a bacterium, virus, or parasite. In some embodiments, the first recombinant microorganism is a bacterium. In some embodiments, the first recombinant microorganism is an enterobacterium, a listeriaceae bacterium, or a streptococcaceae bacterium. In some embodiments, the bacteria is an enterobacterium. In some embodiments, the enterobacterium is a salmonella bacterium. In some embodiments, the salmonella bacterium is a S. bongori or a S. enterica. In some embodiments, the bacteria is a listeriaceae bacterium. In some embodiments, the listeriaceae bacterium is a listeria bacterium. In some embodiments, the listeria bacterium is a L. monocytogenes. In some embodiments, the bacteria is a streptococcaceae bacterium. In some embodiments, the streptococcaceae bacterium is a lactococcus bacteria. In some embodiments, the lactococcus bacteria is a L. chungangensis, L. formosensis, L. fujiensis, L. garvieae, L. hircilactis, L. lactis, L. laudensis, L. nasutitermitis, L. piscium, L. plantarum, L. raffinolactis, or L. taiwanensis. In some embodiments, the first recombinant microorganism is a parasite. In some embodiments, the parasite is a sarcocystidae parasite or a trypanosomatida parasite. In some embodiments, the parasite is a sarcocystidae parasite. In some embodiments, the sarcocystidae parasite is a toxoplasma parasite. In some embodiments, the toxoplasma parasite is Toxoplasma gondii. In some embodiments, the parasite is a trypanosomatida parasite. In some embodiments, the trypanosomatida parasite is a leishmania parasite. In some embodiments, the leishmania parasite is a L. aethiopica, L. amazonensis, L. Arabica, L. aristedesi, L. braziliensis, L. chagasi, L. colombiensis, L. deanei, L. donovani, L. enriettii, L. forattinii, L. garnhami, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, or L. venezuelensis. In some embodiments, the first recombinant microorganism is a virus. In some embodiments, the first recombinant microorganism is of family viridae. In some embodiments, the first recombinant microorganism is a DNA virus. In some embodiments, the first recombinant microorganism is an RNA virus. In some embodiments, the first recombinant microorganism comprises a poxvirus, a picornavirus, an adenovirus, a parvovirus, a herpesvirus, a reovirus, a paramyxovirus, a rhabdovirus, an orthomyxovirus, or a coxsackievirus. In some embodiments, the first recombinant microorganism is a poxvirus. In some embodiments, the poxvirus is an orthopoxvirus, parapoxvirus, yatapoxvirus, a leporipoxvirus, mulluscipoxvirus, a betaentomopoxvirus, a cervidpoxvirus, a gammaentomopoxvirus, a suipoxvirus, a crocodylidpoxvirus, a alphaentomopoxvirus, a capripoxvirus, or an avipoxvirus. In some embodiments, the poxvirus is an orthopoxvirus. In some embodiments, the orthopoxvirus is a vaccinia virus. In some embodiments, the poxvirus is a leporipoxvirus. In some embodiments, the leporipoxvirus is a myxoma virus. In some embodiments, the poxvirus is a picornavirus. In some embodiments, the picornavirus is an aphthovirus, an aquamavirus, an avihepatovirus, an avisivirus, a cardiovirus, a cosavirus, a dicipivirus, an enterovirus, an erbovirus, a gallivirus, an hepatovirus, an hunnivirus, a kobuvirus, a kunsagivirus, a megrivirus, a mischivirus, a mosavirus, an oscivirus, a parechovirus, a pasivirus, a passerivirus, a rosavirus, a sakobuvirus, a salivirus, a sapelovirus, a senecavirus, a sicinivirus, a teschovirus, or a tremovirus. In some embodiments, the picornavirus is an enterovirus. In some embodiments, the enterovirus is a poliovirus or a coxsackievirus. In some embodiments, the picornavirus is a cardiovirus. In some embodiments, the cardiovirus is a mengovirus. In some embodiments, the first recombinant microorganism is an adenovirus. In some embodiments, the adenovirus is an atadenovirus, an aviadenovirus, an ichtadenovirus, a mastadenovirus, or a siadenovirus. In some embodiments, the first recombinant microorganism is a parvovirus. In some embodiments, the parvovirus is an amdoparvovirus, an aveparvovirus, a bocaparvovirus, a chapparvovirus, a copiparvovirus, a dependoparvovirus, an erythroparvovirus, a protoparvovirus, or a tetraparvovirus. In some embodiments, the parvovirus is a dependoparvovirus. In some embodiments, the dependoparvovirus is an adenoassociated virus. In some embodiments, the first recombinant microorganism is a herpesvirus. In some embodiments, the herpesvirus is an alphaherpesvirus, a betaherpesvirius, or a gammaherpesvirius. In some embodiments, the herpesvirus is an alphaherpesvirus. In some embodiments, the alphaherpesvirus is an iltovirus, a mardivirus simplexvirus, or a varicellovirus. In some embodiments, the herpesvirus is a betaherpesvirius. In some embodiments, the betaherpesvirus is a cytomegalovirus, a muromegalovirus, a proboscivirus, or a poseolovirus. In some embodiments, the herpesvirus is a gammaherpesvirius. In some embodiments, the gammaherpesvirius is a lymphocryptovirus, a macavirus, a percavirus, or a rhadinovirus. In some embodiments, the first recombinant microorganism is a rhabdovirus. In some embodiments, the rhabdovirus is a curiovirus, a cytorhabdovirus, a dichorhavirus, an ephemerovirus, a hapavirus, a ledantevirus, a lyssavirus, a novirhabdovirus, a nucleorhabdovirus, a perhabdovirus, a sigmavirus, a sprivivirus, a sripuvirus, a tibrovirus, a tupavirus, a varicosavirus, or a vesiculovirus. In some embodiments, the rhabdovirus is a vesiculovirus. In some embodiments, the vesiculovirus is a vesicular stomatitis virus or a marba virus. In some embodiments, the first recombinant microorganism is a reovirus. In some embodiments, the reovirus is a sedoreovirus or a spinareovirus. In some embodiments, the reovirus is a sedoreovirus.

In some embodiments, the sedoreovirus is a cardoreovirus, a mimoreovirus, an orbivirus, a phytoreovirus, a rotavirus, or a seadornavirus. In some embodiments, the reovirus is a spinareovirus. In some embodiments, the spinareovirus is an aquareovirus, a coltivirus, a cypovirus, a fijivirus, an orthoreovirus, an idnoreovirus, ad dinovernavirus, an oryzavirus, or a mycoreovirus. In some embodiments, the first recombinant microorganism is a paramyxovirus. In some embodiments, the paramyxovirus is an aquaparamyxovirus, an avulavirus, a ferlavirus, an henipavirus, a morbillivirus, a respirovirus, or a rubulavirus. In some embodiments, the paramyxovirus is a morbillivirus. In some embodiments, the morbillivirus is a measles virus. In some embodiments, the first recombinant microorganism is an orthomyxovirus. In some embodiments, the orthomyxovirus is an influenza virus. In some embodiments, the second recombinant microorganism is a bacterium. In some embodiments, the second recombinant microorganism is an enterobacterium, a listeriaceae bacterium, or a streptococcaceae bacterium. In some embodiments, the bacteria is an enterobacterium. In some embodiments, the enterobacterium is a salmonella bacterium. In some embodiments, the salmonella bacterium is a S. bongori or a S. enterica. In some embodiments, the bacteria is a listeriaceae bacterium. In some embodiments, the listeriaceae bacterium is a listeria bacterium. In some embodiments, the listeria bacterium is a L. monocytogenes. In some embodiments, the bacteria is a streptococcaceae bacterium. In some embodiments, the streptococcaceae bacterium is a lactococcus bacteria. In some embodiments, the lactococcus bacteria is a L. chungangensis, L. formosensis, L. fujiensis, L. garvieae, L. hircilactis, L. lactis, L. laudensis, L. nasutitermitis, L. piscium, L. plantarum, L. raffinolactis, or L. taiwanensis. In some embodiments, the second recombinant microorganism is a parasite. In some embodiments, the parasite is a sarcocystidae parasite or a trypanosomatida parasite. In some embodiments, the parasite is a sarcocystidae parasite. In some embodiments, the sarcocystidae parasite is a toxoplasma parasite. In some embodiments, the toxoplasma parasite is Toxoplasma gondii. In some embodiments, the parasite is a trypanosomatida parasite. In some embodiments, the trypanosomatida parasite is a leishmania parasite. In some embodiments, the leishmania parasite is a L. aethiopica, L. amazonensis, L. Arabica, L. aristedesi, L. braziliensis, L. chagasi, L. colombiensis, L. deanei, L. donovani, L. enriettii, L. forattinii, L. garnhami, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, or L. venezuelensis. In some embodiments, the second recombinant microorganism is a virus. In some embodiments, the second recombinant microorganism is of family viridae. In some embodiments, the second recombinant microorganism is a DNA virus. In some embodiments, the second recombinant microorganism is an RNA virus. In some embodiments, the second recombinant microorganism is a poxvirus, a picornavirus, an adenovirus, a parvovirus, a herpesvirus, a reovirus, a paramyxovirus, a rhabdovirus, an orthomyxovirus, or a coxsackievirus. In some embodiments, the second recombinant microorganism is a poxvirus. In some embodiments, the poxvirus is an orthopoxvirus, parapoxvirus, yatapoxvirus, a leporipoxvirus, mulluscipoxvirus, a betaentomopoxvirus, a cervidpoxvirus, a gammaentomopoxvirus, a suipoxvirus, a crocodylidpoxvirus, a alphaentomopoxvirus, a capripoxvirus, or an avipoxvirus. In some embodiments, the poxvirus is an orthopoxvirus. In some embodiments, the orthopoxvirus is a vaccinia virus. In some embodiments, the poxvirus is a leporipoxvirus. In some embodiments, the leporipoxvirus is a myxoma virus. In some embodiments, the poxvirus is a picornavirus. In some embodiments, the picornavirus is an aphthovirus, an aquamavirus, an avihepatovirus, an avisivirus, a cardiovirus, a cosavirus, a dicipivirus, an enterovirus, an erbovirus, a gallivirus, an hepatovirus, an hunnivirus, a kobuvirus, a kunsagivirus, a megrivirus, a mischivirus, a mosavirus, an oscivirus, a parechovirus, a pasivirus, a passerivirus, a rosavirus, a sakobuvirus, a salivirus, a sapelovirus, a senecavirus, a sicinivirus, a teschovirus, or a tremovirus. In some embodiments, the picornavirus is an enterovirus. In some embodiments, the enterovirus is a poliovirus or a coxsackievirus. In some embodiments, the picornavirus is a cardiovirus.

In some embodiments, the cardiovirus is a mengovirus. In some embodiments, the second recombinant microorganism is an adenovirus. In some embodiments, the adenovirus is an atadenovirus, an aviadenovirus, an ichtadenovirus, a mastadenovirus, or a siadenovirus. In some embodiments, the second recombinant microorganism is a parvovirus. In some embodiments, the parvovirus is an amdoparvovirus, an aveparvovirus, a bocaparvovirus, a chapparvovirus, a copiparvovirus, a dependoparvovirus, an erythroparvovirus, a protoparvovirus, or a tetraparvovirus. In some embodiments, the parvovirus is a dependoparvovirus. In some embodiments, the dependoparvovirus is an adenoassociated virus. In some embodiments, the second recombinant microorganism is a herpesvirus. In some embodiments, the herpesvirus is an alphaherpesvirus, a betaherpesvirius, or a gammaherpesvirius. In some embodiments, the herpesvirus is an alphaherpesvirus. In some embodiments, the alphaherpesvirus is an iltovirus, a mardivirus simplexvirus, or a varicellovirus. In some embodiments, the herpesvirus is a betaherpesvirius. In some embodiments, the betaherpesvirus is a cytomegalovirus, a muromegalovirus, a proboscivirus, or a poseolovirus. In some embodiments, the herpesvirus is a gammaherpesvirius. In some embodiments, the gammaherpesvirius is a lymphocryptovirus, a macavirus, a percavirus, or a rhadinovirus. In some embodiments, the second recombinant microorganism is a rhabdovirus. In some embodiments, the rhabdovirus is a curiovirus, a cytorhabdovirus, a dichorhavirus, an ephemerovirus, a hapavirus, a ledantevirus, a lyssavirus, a novirhabdovirus, a nucleorhabdovirus, a perhabdovirus, a sigmavirus, a sprivivirus, a sripuvirus, a tibrovirus, a tupavirus, a varicosavirus, or a vesiculovirus. In some embodiments, the rhabdovirus is a vesiculovirus. In some embodiments, the vesiculovirus is a vesicular stomatitis virus or a marba virus. In some embodiments, the second recombinant microorganism is a reovirus. In some embodiments, the reovirus is a sedoreovirus or a spinareovirus. In some embodiments, the reovirus is a sedoreovirus, In some embodiments, the sedoreovirus is a cardoreovirus, a mimoreovirus, an orbivirus, a phytoreovirus, a rotavirus, or a seadornavirus.

In some embodiments, the reovirus is a spinareovirus. In some embodiments, the spinareovirus is an aquareovirus, a coltivirus, a cypovirus, a fijivirus, an orthoreovirus, an idnoreovirus, ad dinovernavirus, an oryzavirus, or a mycoreovirus. In some embodiments, the second recombinant microorganism is a paramyxovirus. In some embodiments, the paramyxovirus is an aquaparamyxovirus, an avulavirus, a ferlavirus, a henipavirus, a morbillivirus, a respirovirus, or a rubulavirus. In some embodiments, the paramyxovirus is a morbillivirus. In some embodiments, the morbillivirus is a measles virus. In some embodiments, the second recombinant microorganism is an orthomyxovirus. In some embodiments, the orthomyxovirus is an influenza virus. In some embodiments, the first recombinant microorganism comprises an exogenous nucleic acid encoding one or more human proteins and the second recombinant microorganism does not comprise an exogenous nucleic acid encoding the one or more human proteins. In some embodiments, the human protein is a chemokine or cytokine. In some embodiments, the second recombinant microorganism comprises an exogenous nucleic acid encoding one or more human protein and the second recombinant microorganism does not comprise an exogenous nucleic acid encoding the one or more human protein. In some embodiments, the human protein is a chemokine or cytokine. In some embodiments, the subject is a human. In some embodiments, the tumor is a solid tumor or a hematological tumor. In some embodiments, the tumor comprises a prostate tumor, lung tumor, renal tumor, stomach tumor, colon tumor, ovarian tumor, bladder tumor, breast tumor, cervical tumor, esophageal tumor, testicular tumor, liver tumor, pancreatic tumor, rectal tumor, thyroid tumor, uterine tumor, skin tumor, muscle tumor, cartilage tumor, bone tumor, endothelial tumor, epithelial tumor, leukemia, lymphoma, myeloma, dermal tumor, basal tumor, retinal tumor, skin tumor, or brain tumor. In some embodiments, the first recombinant microorganism and the second recombinant microorganism are administered to the subject simultaneously. In some embodiments, the first recombinant microorganism is formulated in a delayed release composition, a sustained release composition, an immediate release composition, a stealth release composition, or any combinations thereof. In some embodiments, the second recombinant microorganism is formulated in a delayed release composition. In some embodiments, the second recombinant microorganism is administered to the subject after the first recombinant microorganism is administered to the subject. In some embodiments, the second recombinant microorganism is administered from about 1-60 days, from 1-45 days, from 1-30 days, from 1-15 days, from 1-10 days, or from 1-7 days after administration of the first recombinant microorganism.

In some embodiments, the second recombinant microorganism is administered about 1, 2, 3, 4, 5, 6, 7, 8, 9, 10, 11, 12, 14, 21, 28, 35, 56, or 60 days after administration of the first recombinant microorganism. In some embodiments, the second recombinant microorganism is administered to the subject one, two, three, three, four, five or more times. In some embodiments, the second recombinant microorganism is administered to the subject two, three, three, four, five or more times with about 1-60 days, 1-45 days, 1-30 days, 1-15 days, 1-10 days, 1-7, 1-5 days, or 1-3 days between each administration. In some embodiments, the first recombinant microorganism is administered to the subject after the second recombinant microorganism is administered to the subject.

In some embodiments, the first recombinant microorganism is administered from about 1-60 days, from 1-45 days, from 1-30 days, from 1-15 days, from 1-10 days, or from 1-7 days after administration of the second recombinant microorganism.

In some embodiments, the first recombinant microorganism is administered about 1, 2, 3, 4, 5, 6, 7, 8, 9, 10, 11, 12, 14, 21, 28, 35, 56, or 60 days after administration of the second recombinant microorganism. In some embodiments, the first recombinant microorganism is administered to the subject one, two, three, three, four, five or more times. In some embodiments, the first recombinant microorganism is administered to the subject two, three, three, four, five or more times with about 1-60 days, 1-45 days, 1-30 days, 1-15 days, 1-10 days, 1-7, 1-5 days, or 1-3 days between each administration. In some embodiments, the first recombinant microorganism is administered intra-tumorally, intradermally, subcutaneously, intraperitoneally, intramuscularly or intravenously; and the second composition is administered intra-tumorally, intradermally, subcutaneously, intraperitoneally, intrathecally, intramuscularly or intravenously. In some embodiments, the second recombinant microorganism is administered intra-tumorally, intradermally, subcutaneously, intraperitoneally, intramuscularly; and the second composition is administered intra-tumorally, intradermally, subcutaneously, intraperitoneally, intrathecally, intramuscularly or intravenously.

In some embodiments, the method further comprises administering an anti-cancer therapy. In some embodiments, the anti-cancer therapy comprises chemotherapy, radiation, an immunomodulatory agent, or a cell therapy. In some embodiments, the immunomodulatory agent comprises an immune checkpoint inhibitor. In some embodiments, the immune checkpoint inhibitor comprises a protein that binds to PD-1, PD-L1, CTLA-4, A2AR, B7-H3, B7-H4, BTLA, IDO, KIR, LAG3, TIM-3, VISTA, CD160, TIGIT, PSGL-1, or any combinations thereof. In some embodiments, the immune checkpoint inhibitor comprises an antibody that specifically binds to PD-1, PD-L1, CTLA-4, A2AR, B7-H3, B7-H4, BTLA, IDO, KIR, LAG3, TIM-3, VISTA, CD160, TIGIT, PSGL-1, or any combinations thereof.

In some embodiments, the immune response is demonstrated by the change in population of immune cells, wherein the immune cells comprise myeloid derived suppressor cells, regulatory T cells, natural killer cells, dendritic cells, or any combinations thereof. In some embodiments, the immune response is demonstrated by the enhancement in cytokine production, wherein the cytokine comprises IFN-γ, IL-12, TNFα, or any combinations thereof. In some embodiments, the method further comprises administering to the subject a further recombinant microorganism. In some embodiments, the method further comprises administering to the subject a third recombinant microorganism. In some embodiments, the method further comprises administering to the subject a fourth recombinant microorganism.

One embodiment provides a kit comprising at least a first and a second container and instructions for use, wherein the first container comprises a first recombinant microorganism and the second container comprises a second recombinant microorganism. In some embodiments, the first recombinant microorganism replicates in a tumor cell and does not replicate in a non-tumor cell or displays attenuated replication in a non-tumor cell and wherein the second recombinant microorganism replicates in a tumor cell and does not replicate in a non-tumor cell or displays attenuated replication in a non-tumor cell. In some embodiments, the kit further comprises a third container comprising a third recombinant microorganism. In some embodiments, the kit further comprises a fourth container comprising a fourth recombinant microorganism.

In some embodiments, the third recombinant microorganism replicates in a tumor cell and does not replicate in a non-tumor cell or displays attenuated replication in a non-tumor cell and wherein the fourth recombinant microorganism replicates in a tumor cell and does not replicate in a non-tumor cell or displays attenuated replication in a non-tumor cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various features of this disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 shows the therapeutic activity of a heterologous prime-boost regimen of Salmonella bacterium and Vaccinia virus in mouse renal adenocarcinoma (RENCA) tumor models.

FIG. 2A and FIG. 2B show the therapeutic activity of a heterologous prime-boost regimen of Vaccinia virus or Herpes simplex virus or Vesicular stomatitis virus in mouse RENCA tumor models.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous embodiments and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

### I. Certain Definitions

The following definitions are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

A The terminology used herein is for the purpose of describing particular cases only and is not intended to be limiting. As used herein, the singular forms “a”, “an” and “the” include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “individual,” “patient,” or “subject” are used interchangeably herein. None of the terms require or are limited to situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker. The term “subject” can refer to an animal, including, but not limited to, a primate (e.g., human), cow, sheep, goat, horse, dog, cat, rabbit, rat, or mouse.

The terms “inhibiting,” “reducing” or “prevention,” or any variation of these terms, referred to herein, include any measurable decrease or complete inhibition to achieve a desired result.

The terms “treat,” “treating,” and “treatment” include alleviating or abrogating a disorder, disease, or condition; or one or more of the symptoms associated with the disorder, disease, or condition; or alleviating or eradicating the cause(s) of the disorder, disease, or condition itself. Desirable effects of treatment include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The terms “systemic delivery,” and “systemic administration,” used interchangeably herein, refer to a route of administration of medication, recombinant microorganisms or other substances into the circulatory system. The systemic administration may comprise oral administration, parenteral administration, intranasal administration, sublingual administration, rectal administration, transdermal administration, or any combination thereof.

The term “therapeutically effective amount” refers to the amount of an agent (e.g., recombinant microorganism described herein) that, when administered, is sufficient to prevent development of, or alleviate to some extent, one or more of the symptoms of the disorder, disease, or condition being treated. The term “therapeutically effective amount” can also refer to the amount of an agent (e.g., recombinant microorganism) that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component is “pharmaceutically acceptable” in the sense of being compatible with the other ingredients of a pharmaceutical formulation. It is also suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of an agent (e.g., recombinant microorganism described herein) disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The terms “recombinant microorganism,” as used interchangeably herein, refer to a microorganism, e.g., a virus, that is genetically modified by experimental intervention. For example, a recombinant microorganism comprises one or more mutations in its genome, including but not limited to deletions, insertions of heterologous nucleic acids, inversions, substitutions or combinations thereof.

The term “naturally-occurring,” as used herein with reference to a microorganism, e.g., a virus, indicates that the microorganisms can be found in nature, i.e., it can be isolated from a source in nature and has not been intentionally modified.

The term “attenuated” as used herein can mean that the recombinant microorganism has a reduced ability to infect a non-cancer cell relative to the ability of the recombinant microorganism to infect a cancer cell; a reduced ability to replicate in a non-cancer cell relative to the ability of the recombinant microorganism to replicate in a cancer cell; a reduced replication efficiency in a non-cancer cell relative to the replication efficiency in a cancer cell; or a reduced ability lyse a non-cancer cell relative to the ability to lyse a cancer cell. In some cases, the cancer cell and the non-cancer cell can be of the same type, e.g., an endothelial cell, an epithelial cell. In some cases, the cancer cell and the non-cancer cell can be of different types, e.g., in some cases the cancer cell can be an endothelial cell and the non-cancer cell can be an epithelial cell.

The term “serologically distinct” as used herein with reference to two or more different microorganisms indicates that there may be no or reduced cross-reactivity between antibodies generated by a subject to the two or more different microorganisms.

The term “tumor associated cell” as used herein can refer to non-tumorous cells present within the microenvironment of a solid tumor in a subject. These cells can include, for example, fibroblasts, endothelial cells, stromal cells, and immune cells.

The term “tumor protein” as used herein can refer to a protein that is expressed at an increased level or in a mutated form (or both) by a tumor cell prior to infection with a recombinant microorganism described herein relative to the level of expression or form expressed by a cell of the same type that is not tumorous.

### II. Recombinant Microorganisms

Recombinant microorganisms described herein include, but are not limited to, viruses, bacteria, and parasites. In some embodiments, the recombinant microorganism can be a virus. In some embodiments, the recombinant microorganism can be a bacterium. In some embodiments, the recombinant microorganism can be a parasite.

In some embodiments, the first recombinant microorganism can be a virus and the second recombinant microorganism can be a different virus. In some embodiments, the first recombinant microorganism can be a virus and the second recombinant microorganism can be a bacterium. In some embodiments, the first recombinant microorganism can be a virus and the second recombinant microorganism can be a parasite.

In some embodiments, the second recombinant microorganism can be a virus and the first recombinant microorganism can be a different virus. In some embodiments, the second recombinant microorganism can be a virus and the first recombinant microorganism can be a bacterium. In some embodiments, the second recombinant microorganism can be a virus and the first recombinant microorganism can be a parasite.

In some embodiments, the recombinant microorganism can be a bacterium. In some embodiments, the recombinant microorganism can be an enterobacterium, a listeriaceae bacterium, or a streptococcaceae bacterium. In some embodiments, the bacteria can be an enterobacterium. In some embodiments, the enterobacterium can be a salmonella bacterium. In some embodiments, the salmonella bacterium can be an attenuated Salmonella Typhimurium, such as auxotrophic (aroA mutant). In some embodiments, the salmonella bacterium can be a S. bongori or a S. enterica. In some embodiments, the bacteria can be a listeriaceae bacterium. In some embodiments, the listeriaceae bacterium can be a listeria bacterium. In some embodiments, the listeria bacterium can be a L. monocytogenes. In some embodiments, the bacteria can be a streptococcaceae bacterium. In some embodiments, the streptococcaceae bacterium can be a lactococcus bacteria. In some embodiments, the lactococcus bacteria can be a L. chungangensis, L. formosensis, L. fujiensis, L. garvieae, L. hircilactis, L. lactis, L. laudensis, L. nasutitermitis, L. piscium, L. plantarum, L. raffinolactis, or L. taiwanensis.

In some embodiment, the recombinant microorganism can be a parasite. In some embodiments, the parasite can be a sarcocystidae parasite or a trypanosomatida parasite. In some embodiments, the parasite can be sarcocystidae parasite. In some embodiments, the sarcocystidae parasite can be a toxoplasma parasite. In some embodiments, the toxoplasma parasite can be a Toxoplasma gondii. In some embodiments, the parasite can be a trypanosomatida parasite.

In some embodiments, the trypanosomatida parasite is a leishmania parasite. In some embodiments, the leishmania parasite can be a L. aethiopica, L. amazonensis, L. Arabica, L. aristedesi, L. braziliensis, L. chagasi, L. colombiensis, L. deanei, L. donovani, L. enriettii, L. forattinii, L. garnhami, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, or L. venezuelensis.

In some embodiments, the recombinant microorganism can be a virus. In some embodiments, the first recombinant microorganism can be of family viridae. In some embodiments, the recombinant microorganism can be a DNA virus. In some embodiments, the recombinant microorganism can be an RNA virus.

In some embodiments, the recombinant microorganism can be a poxvirus, a picornavirus, an adenovirus, a parvovirus, a herpesvirus, a reovirus, a paramyxovirus, a rhabdovirus, an orthomyxovirus, or a coxsackievirus.

In some embodiments, the recombinant microorganism can be a poxvirus. In some embodiments, the poxvirus can be an orthopoxvirus, parapoxvirus, yatapoxvirus, a leporipoxvirus, mulluscipoxvirus, a betaentomopoxvirus, a cervidpoxvirus, a gammaentomopoxvirus, a suipoxvirus, a crocodylidpoxvirus, a alphaentomopoxvirus, a capripoxvirus, or an avipoxvirus. In some embodiments, the poxvirus can be an orthopoxvirus. In some embodiments, the orthopoxvirus can be a vaccinia virus. In some embodiments, the poxvirus can be a leporipoxvirus. In some embodiments, the leporipoxvirus can be a myxoma virus.

In some embodiments, the poxvirus can be a picornavirus. In some embodiments, the picornavirus can be an aphthovirus, an aquamavirus, an avihepatovirus, an avisivirus, a cardiovirus, a cosavirus, a dicipivirus, an enterovirus, an erbovirus, a gallivirus, an hepatovirus, an hunnivirus, a kobuvirus, a kunsagivirus, a megrivirus, a mischivirus, a mosavirus, an oscivirus, a parechovirus, a pasivirus, a passerivirus, a rosavirus, a sakobuvirus, a salivirus, a sapelovirus, a senecavirus, a sicinivirus, a teschovirus, or a tremovirus. In some embodiments, the picornavirus is an enterovirus. In some embodiments, the enterovirus can be a poliovirus or a coxsackievirus. In some embodiments, the picornavirus can be a cardiovirus. In some embodiments, the cardiovirus can be a mengovirus.

In some embodiments, the microorganism can be an adenovirus. In some embodiments, the adenovirus can be an atadenovirus, an aviadenovirus, an ichtadenovirus, a mastadenovirus, or a siadenovirus.

In some embodiments, the recombinant microorganism can be a parvovirus. In some embodiments, the parvovirus can be an amdoparvovirus, an aveparvovirus, a bocaparvovirus, a chapparvovirus, a copiparvovirus, a dependoparvovirus, an erythroparvovirus, a protoparvovirus, or a tetraparvovirus. In some embodiments, the parvovirus can be a dependoparvovirus. In some embodiments, the dependoparvovirus can be an adenoassociated virus.

In some embodiments, the recombinant microorganism can be a herpesvirus. In some embodiments, the herpesvirus can be an alphaherpesvirus, a betaherpesvirius, or a gammaherpesvirius. In some embodiments, the herpesvirus can be an alphaherpesvirus. In some embodiments, the alphaherpesvirus can be an iltovirus, a mardivirus simplexvirus, or a varicellovirus. In some embodiments, the herpesvirus can be a betaherpesvirius. In some embodiments, the betaherpesvirus can be a cytomegalovirus, a muromegalovirus, a proboscivirus, or a poseolovirus. In some embodiments, the herpesvirus can be a gammaherpesvirius. In some embodiments, the gammaherpesvirius can be a lymphocryptovirus, a macavirus, a percavirus, or a rhadinovirus.

In some embodiments, the recombinant microorganism can be a rhabdovirus. In some embodiments, the rhabdovirus can be a curiovirus, a cytorhabdovirus, a dichorhavirus, an ephemerovirus, a hapavirus, a ledantevirus, a lyssavirus, a novirhabdovirus, a nucleorhabdovirus, a perhabdovirus, a sigmavirus, a sprivivirus, a sripuvirus, a tibrovirus, a tupavirus, a varicosavirus, or a vesiculovirus In some embodiments, the rhabdovirus can be a vesiculovirus. In some embodiments, the vesiculovirus can be a vesicular stomatitis virus or a marba virus.

In some embodiments, the recombinant microorganism can be a reovirus. In some embodiments, the reovirus can be a sedoreovirus or a spinareovirus. In some embodiments, the reovirus can be a sedoreovirus. In some embodiments, the sedoreovirus can be a cardoreovirus, a mimoreovirus, an orbivirus, a phytoreovirus, a rotavirus, or a seadornavirus. In some embodiments, the reovirus can be a spinareovirus. In some embodiments, the spinareovirus can be an aquareovirus, a coltivirus, a cypovirus, a fijivirus, an orthoreovirus, an idnoreovirus, ad dinovernavirus, an oryzavirus, or a mycoreovirus.

In some embodiments, the recombinant microorganism can be a paramyxovirus. In some embodiments, the paramyxovirus can be an aquaparamyxovirus, an avulavirus, a ferlavirus, an henipavirus, a morbillivirus, a respirovirus, or a rubulavirus. In some embodiments, the paramyxovirus can be a morbillivirus. In some embodiments, the morbillivirus can be a measles virus.

In some embodiments, the recombinant microorganism can be an orthymuxovirus. In some embodiments, the orthomyxovirus is an influenza virus.

In some embodiments, the recombinant microorganism is oncolytic. As used herein, the term “oncolytic microorganism,” refers to a microorganism that preferentially replicates in, infects, or kills cancer cells relative to non-cancer cells. Under certain non-limiting circumstances, it is understood that oncolytic microorganisms (e.g., oncolytic viruses) can promote anti-cancer responses through dual mechanisms dependent on not only the selective killing of tumor cells, but also the stimulation of host anti-tumor immune responses.

In some embodiments, the recombinant microorganism replicates in cancer cells and is attenuated or does not replicate in non-cancer cells. In some embodiments, recombinant microorganism replicates in cancer cells, and does not replicate in non-cancer cells. In some embodiments, the recombinant microorganism replicates in cancer cells, and is attenuated in non-cancer cells. In some embodiments, the recombinant microorganism mediates lysis of a plurality of cancer cells in vivo. In some embodiments, the recombinant microorganism mediates lysis of a plurality of cancer cells in vivo, and does not replicate in non-cancer cells. In some embodiments, the recombinant microorganism mediates lysis of a plurality of cancer cells in vivo, and is attenuated in non-cancer cells.

In some embodiments, recombinant oncolytic microorganisms can include, but are not limited to, (i) recombinant microorganisms that naturally replicate preferentially in cancer cells and are non-pathogenic in humans often due to elevated sensitivity to innate anti-microorganism signaling or dependence on oncogenic signaling pathways; and (ii) recombinant microorganisms that are genetically-manipulated for use.

The infectivity of a recombinant microorganism described herein can be determined using standard methods known in the art. For example, viral infectivity can be measured using a viral plaque assay, fluorescent focus assay (FFA), endpoint dilution assay (TCID50), qPCR to determine the level of viral RNA or DNA, ELISA to detect specific viral proteins, and transmission electron microscopy to count virus particles.

Replication of a recombinant microorganism described herein can be determined using standard methods known in the art. For example, viral replication can be determined using standard plaque assay techniques that are known in the art. For example, a population of cells can be infected with virus, and supernatant collected and analyzed for viral titer at various time points post-infection. These assays can be used to establish a semi-quantitative measure of relative viral replication in non-cancer cells (e.g., described herein) versus cancerous cells (e.g., cancerous cells described herein), i.e., the ratio of replication. Larger ratios are indicative of viruses that replicated more efficiently in cancer cells than in non-cancer cells.

In some embodiments, the recombinant microorganism can comprise an exogenous nucleic acid encoding a pro-inflammatory protein or a functional domain thereof or a fragment thereof. In some embodiments, the pro-inflammatory protein can be a chemokine or cytokine. In some embodiments, the recombinant microorganism can comprise an exogenous nucleic acid encoding a receptor of a pro-inflammatory protein (e.g., a pro-inflammatory cytokine or chemokine).

Exemplary pro-inflammatory cytokines and chemokines can include, but are not limited to, tumor necrosis factors (TNF) (e.g., TNF-α, TNF-β), TNF superfamily molecules (e.g., FasL, CD27L, CD30L, CD40L, Ox40L, 4-1BBL, TRAIL, TWEAK, Apo3L), IL-1 (e.g., IL-1α and IL-1β), IL-2, interferon-γ (IFN-γ), IFN-α/β, IL-6, IL-8, IL-12, IL-15, IL-17, IL-18, IL-21, LIF, CCL5, GROα, MCP-1, MIP-1α, MIP-1β, MCSF, GM-CSF, CXCL2, CCL2, and RANTES.

In some embodiments, the recombinant microorganism can comprise an exogenous nucleic acid encoding an anti-inflammatory protein or a functional domain thereof or a fragment thereof. In some embodiments, the anti-inflammatory protein can be a chemokine or cytokine. In some embodiments, the recombinant microorganism can comprise an exogenous nucleic acid encoding a receptor of an anti-inflammatory protein (e.g., an anti-inflammatory cytokine or chemokine). Exemplary anti-inflammatory cytokines and chemokines can include, but are not limited to, IL-4, IL-6, IL-10, IL-11, IL-13, and TGFβ.

In some embodiments, the recombinant microorganism can comprise an exogenous nucleic acid encoding a cytokine receptor whose cognate cytokine can be expressed in tumor microenvironments (e.g., IL15-R can have a cognate cytokine IL15 expressed in a tumor microenvironment). In some embodiments, the recombinant microorganism can express selected chemokine receptors whose cognate chemokines are likely to be expressed on tumors (e.g., CXCR4 can have a cognate chemokine CXCL12 expressed on a tumor; CCR2 can have a target CCL2 expressed on a tumor) and can be delivered systemically. Non-limiting examples of chemokine receptors, as described herein can include, but are not limited to, CXC chemokine receptors, CC chemokine receptors, CX3C chemokine receptors and XC chemokine receptors that correspond to the 4 distinct subfamilies of chemokines they bind. Non-limiting embodiments of the present disclosure provide a recombinant microorganism that can comprise an exogenous nucleic acid that encodes a chemokine receptor. In some embodiments, the chemokine receptor is a CXC chemokine receptor, a CC chemokine receptor, a CX3C chemokine receptor, a XC chemokine receptor, or any combination thereof. In some embodiments, the chemokine receptor can be CXCR1, CXCR2, CXCR2, CXCR3, CXCR4, CXCR5, CXCR6, CXCR7, CCR1, CCR2, CCR3, CCR4, CCR5, CCR6, CCR7, CX3CR1, XCR1, or any combination thereof.

In some embodiments, the recombinant microorganism can comprise an exogenous nucleic acid that encodes a chemokine receptor that is a chimeric protein. In some embodiments, at least part of its extracellular domain can be from a chemokine receptor that promotes the tumor-targeted delivery of the recombinant microorganism, and at least part of its intracellular domain is from a chemokine receptor that promotes the tumor-specific replication, inhibits immunosuppressive activity, or conveys some other beneficial effects, or vice versa. For instance, in some embodiments, the recombinant microorganism can comprise a nucleic acid that codes for a protein having an intracellular GTPase domain of CCR5, and an extracellular chemokine-binding domain of CXCR4 or CCR2. In some embodiments, by combining domains with different functionalities one achieves further improvement in therapeutic performance of the recombinant microorganism.

In some embodiments, the recombinant microorganism can comprise one or more exogenous nucleic acid that encode at least one chemokine receptor. In some embodiments, the recombinant microorganisms can comprise exogenous nucleic acids that encode two or more different chemokine receptors, which may be expressed simultaneously by the recombinant microorganism. Exemplary chemokine receptors that can be expressed simultaneously from the recombinant microorganism described herein include CXCR4 and CCR2. In recombinant microorganisms expressing more than one chemokine receptors, a combinatorial or synergistic effect against tumor cells may be achieved as to the therapeutic application of the recombinant microorganism.

In some embodiments, the modification of the recombinant microorganism can result in at least about 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 7.5×10⁵, 10⁶, 2.5×10⁶, 5×10⁶, 7.5×10⁶, 10⁷, 2.5×10⁷, 5×10⁷, 7.5×10⁷, 10⁸, 2.5×10⁸, 5×10⁸, 7.5×10⁸, 10⁹, 2.5×10⁹, 5×10⁹, 7.5×10⁹, 10¹⁰ or more fold increase in the efficacy of tumor-targeted systemic delivery of the recombinant microorganism, as compared to an otherwise identical recombinant microorganism that does not comprise the modification.

In some embodiments, the recombinant microorganism can comprise an exogenous nucleic acid that encodes a chemokine receptor and the forced expression of chemokine receptor by the recombinant microorganism results in boosted immune responses against the infected tumor. Following infecting the tumor, the recombinant microorganism can replicate in the tumor cells and result in the expression of the chemokine receptors on the surface of the tumor cells. These membrane receptors may function as decoy receptors, binding and sequestering the immunosuppressive chemokines within the tumor. Consequently, the immunosuppressive microenvironment in the tumor can be altered, leading to enhanced immunotherapeutic activity of the recombinant microorganism, as compared to an otherwise identical microorganism that does comprise the nucleic acid coding for the chemokine receptor. In some embodiments, the increase in immunotherapeutic activity is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or higher fold increases. Without being limited, the increased immunotherapeutic activity can be reflected by increased B cell accumulation in the tumor, increased T cell response to tumor-related immunogens, or both. B cell accumulation can be measured, for example, by quantifying the B cells in the tumor, and T cell immuno-activity may be measured by, for example, interferon-γ (interferon-gamma) secretion in ELISPOT assays, ELISA, co-culture assays, flow cytometry, or any combination thereof.

In some embodiments, the recombinant microorganism comprises an exogenous nucleic acid that encodes a chemokine receptor and the forced expression of chemokine receptor by the recombinant results in increased replication of the microorganism in tumor cells, as compared to an otherwise identical microorganism that does not comprise the nucleic acid coding for the chemokine receptor. In certain embodiments, the recombinant microorganism comprises an exogenous CCR2-expressing nucleic acid, which increases the tumor-specific replication of the recombinant microorganism. In some embodiments, the recombinant microorganism comprises an exogenous CCR5-expressing nucleic acid, which increases the tumor-specific replication of the microorganism. In some embodiments, the increase in tumor-specific replication is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or higher fold increases. Exemplary methods for measuring the increase in viral delivery and spread in tumors include, but are not limited to, fluorescence or bioluminescence-based imaging of expression of a reporter gene, quantitative PCR for detection of tumor concentrations of microbial genomes (e.g., viral genomes) or plaque determination of plaque forming units or immunohistochemistry of microbial protein (e.g., viral proteins).

In some embodiments, the recombinant microorganism can comprise an exogenous nucleic acid that encodes a protein that degrades ECM of a tumor. Exemplary proteins that degrade ECM can include, but are not limited to, membrane associated proteins. In some embodiments, the membrane associated protein can comprise a glycosylphosphatidylinositol (GPI) anchor.

Hyaluronidases are a family of enzymes that catalyze the degradation of Hyaluronan (HA). There are at least five functional hyaluronidases identified in humans: HYAL1, HYAL2, HYAL3, HYAL4 and HYAL5 (also known as PH-20 or SPAM1), among which PH-20 is the only one known to be functional at relatively neutral pH. In some embodiments of the present disclosure, combining hyaluronidase with other tumor-targeting therapeutic agents (such as transgenes, also referred to herein as exogenous nucleic acid) can promote the therapeutic effect of the recombinant microorganism at least by diminishing the ECM and enhancing the transportation of the therapeutic agent inside and between the tumors.

In some embodiments, the recombinant microorganism can comprise an exogenous nucleic acid coding for a membrane associated protein that is capable of degrading hyaluronan, such as a hyaluronidase. The term “hyaluronidase” as used herein refers to any enzyme or a fragment thereof that catalyzes the degradation of HA in a tumor, including, but not limited to, PH-20 and its homologs from other species, as well as other engineered/design proteins with similar enzymatic function. Hyaluronidase includes class of hyaluronan degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases can be of any non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases can include, hyaluronidases from cows, yellow jacket wasp, honey bee, white-face hornet, paper wasp, mouse, pig, rat, rabbit, sheep, chimpanzee, Rhesus monkey, orangutan, cynomolgus monkey, guinea pig, Arthrobacter sp. (strain FB24), Bdellovibrio bacteriovorus, Propionibacterium acnes, Streptococcus agalactiae, Staphylococcus aureus; strain MRSA252, Streptococcus pneumoniae, Streptococcus pyogenes, Streptococcus suis, Vibrio fischeri, and the Streptomyces hyaluronolyticus hyaluronidase enzyme, which is specific for hyaluronic acid and does not cleave chondroitin or chondroitin sulfate.

In some embodiments, the recombinant microorganism can comprise an exogenous nucleic acid that encodes a secreted hyaluronidase. In some examples, the secreted hyaluronidase is of microbial origin, such as HysA from Staphylococcus aureus, lin, sko from Saccoglossus kowalevskii, rv. In some embodiments, the exogenous nucleic acid hysA can comprise a genomic sequence corresponding to GenBank sequence Accession No. U21221.1 (SEQ ID NO: 1). In some embodiments, the exogenous nucleic acid hysA encodes a protein HysA that comprises an amino acid sequence as set forth in SEQ ID NO: 2 (corresponding to Uniprot Accession No. UniProtKB-Q59801 (HYSA_STAA8)). In some embodiments, expression of a secreted hyaluronidase, such as HysA, enhances replication, spread, therapeutic activity (e.g., cancer cell killing potential) of recombinant microorganism as described herein.

Furthermore, in some embodiments, the recombinant microorganism can comprise one or more exogenous nucleic acids that encode both a hyaluronidase and a matrix metalloprotease. Collectively, matrix metalloproteases are capable of degrading all kinds of ECM proteins. Examples can include, but are not limited to, MMP1, MMP2, MMP3, MMP7, MMP8, MMP9, MMP10, MMP11, MMP12, MMP13, MMP14, MMP15, MMP17, MMP18, MMP19, MMP20, MMP21, MMP23A, MMP23B, MMP24, MMP25, MMP26, MMP27, and MMP28.

In some embodiments, the recombinant microorganism comprises a nucleic acid encoding a protein that degrades ECM of a tumor increases microorganism spreading in and between tumors as compared to an otherwise identical microorganism that does not comprise the nucleic acid encoding a protein that degrades ECM of a tumor. In some embodiments, such increase is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher fold. Exemplary methods for measuring the increase in microbial spread can include, but are not limited to, fluorescence or bioluminescence-based imaging of expression of a reporter gene, quantitative PCR for detection of tumor concentrations of microbial genomes (e.g., viral genomes) or plaque determination of plaque forming units or immunohistochemistry of microbial protein (e.g., viral proteins).

In some embodiments, the recombinant microorganism does not comprise an exogenous nucleic acid encoding a tumor protein as described herein.

In some embodiments, the tumor protein can include, but is not limited to, mesothelin, BCMA, HER2, GD2, CD19, CD20, CD22, CD30, CD33, CD123, CD38, CD44, CD70, CD274, CD45, CD123, CD138, CD171, ROR1, EGFR, EphA2, FBP, FAP, CEA, EGP2, EGP40, TAG72, PSMA, PSA, PAP, hsp70-2, M-CSF, LAGE-1a, p53, NKG2D ligand, B7-H6, IL-13 R α 2, IL-11 R α, MUC1, MUC16, CA9, GD3, HMW-MAA, CD171, Lewis Y, G250/CAIX, HLA-AI MAGE A1, HLA-A2 NY-ESO-1, PSC1, PCTA-1, MAGE, ELF2M, IGF-I, IGF-II, IGF-I receptor, hTERT, WT1, MUC1, LMP2, HPV16, HPV18, RGL4, MelanA, MART, ML-IAP, AFP, BCR, ABL, CYP1B1, PLAC1, BORIS, NY-BR-1, RGS5, SART3, EphA2, Glypican-3, 5T4, 8H9, αvβ6 integrin, B7-H3, B7-H6, CAIX, CA9, CSPG4, EGP2, EGP40, EPCAM, ERBB3, ERBB4, ErbB3/4, FAP, FAR, FBP, KDR, MCSP, Muc1, Muc16, NCAM, PRAIVIE, ROR1, CD44v7/8, 8H9, NCAM, VEGF-R, TAG72, RAGE-1, MN-CA IX, RU1, RU2 (AS), fetal AchR, TEM1, TEM8, PAX5, OY-TES1, LCK, HMWMAA, AKAP-4, SSX2, XAGE 1, tie 2, PDGFR-β, kallikrein 4, PBF, PRAIVIE, HSDL1, CA125, TADG-12, MUC16, mannan-MIC-1, HERV-K-MEL, KK-LC-1, KM-HN-1, LAGE-1, MAGE-A4, SP17, SSX4, TAG1, TAG2, ENAH, mammaglobin-A, NY-BR-1, BAGE-1, HERV-K-MEL, KK-LC-1, KM-KN-1, LAGE1, MAGE1A, MAGEA2, mucink, TRAG3, c-myc, cyclin B1, p62, DKK1, RU2AS, k-ras, ME1, NFYC, STEAP1, FGF5, RU2AS, hsp70-2, ARTC1, B-RAF, beta-catenin, CDC27, CDK4, CDK12, CDKN2A, CLPP, CSNK1A1, FN1, GAS7, GPNMB, HAUS3, LDLR-fucosyltransferase, MART2, MATN, MUM1, MUM2, MUM3, neo-PAP, myosin, PPP1R3B, PRDX5, PTPRK, RBAF600, SIRT2, SNRPD1, trioephosphate isomerase, OA1, RAB38, TRP1, TRP2, melan-A, BAGE1, GAGE1, GAGE2, GAGE8, GAGE3, GAGE4, GAGE5, GAGE6, GAGE7, GNTVF, LY6K, TRAG3, CASP8, SAGE, DEK-CAN, EFTUD2, FLT3-ITD, cyclin A1, FNDC3B, MAGEAG, G250, hepsin, intestinal carboxyl esterase, PBF, CASP5, COA1, OGT, OS9, CALCA, MDM2, alpha actinin4, elongation factor 2, fos-related antigen 1, legumain, sperm protein 17, carbonic anhydrase IX, folate receptor-α, neutrophil elastase, ephrinB2, glioma-associated antigen, β-human chorionic gonadotropin, alphafetoprotein thyroglobulin, telomerase reverse transcriptase, intestinal carboxy esterase, prostein, survivin, and any function portion thereof.

In some embodiments, the recombinant microorganism does not comprise one or more exogenous nucleic acid encoding one or more of the following human proteins or an antigen thereof: mesothelin, BCMA, HER2, GD2, CD19, CD20, CD22, CD30, CD33, CD123, CD38, CD44, CD70, CD274, CD45, CD123, CD138, CD171, ROR1, EGFR, EphA2, FBP, FAP, CEA, EGP2, EGP40, TAG72, PSMA, PSA, PAP, hsp70-2, M-CSF, LAGE-1a, p53, NKG2D ligand, B7-H6, IL-13 R α 2, IL-11 R α, MUC1, MUC16, CA9, GD3, HMW-MAA, CD171, Lewis Y, G250/CAIX, HLA-AI MAGE A1, HLA-A2 NY-ESO-1, PSC1, PCTA-1, MAGE, ELF2M, IGF-I, IGF-II, IGF-I receptor, hTERT, WT1, MUC1, LMP2, HPV16, HPV18, RGL4, MelanA, MART, ML-IAP, AFP, BCR, ABL, CYP1B1, PLAC1, BORIS, NY-BR-1, RGS5, SART3, EphA2, Glypican-3, 5T4, 8H9, αvβ6 integrin, B7-H3, B7-H6, CAIX, CA9, CSPG4, EGP2, EGP40, EPCAM, ERBB3, ERBB4, ErbB3/4, FAP, FAR, FBP, KDR, MCSP, Muc1, Muc16, NCAM, PRAME, ROR1, CD44v7/8, 8H9, NCAM, VEGF-R, TAG72, RAGE-1, MN-CA IX, RU1, RU2 (AS), fetal AchR, TEM1, TEM8, PAX5, OY-TES1, LCK, HMWMAA, AKAP-4, SSX2, XAGE 1, tie 2, PDGFR-β, kallikrein 4, PBF, PRAME, HSDL1, CA125, TADG-12, MUC16, mannan-MIC-1, HERV-K-MEL, KK-LC-1, KM-HN-1, LAGE-1, MAGE-A4, SP17, SSX4, TAG1, TAG2, ENAH, mammaglobin-A, NY-BR-1, BAGE-1, HERV-K-MEL, KK-LC-1, KM-KN-1, LAGE1, MAGE1A, MAGEA2, mucink, TRAG3, c-myc, cyclin B1, p62, DKK1, RU2AS, k-ras, ME1, NFYC, STEAP1, FGF5, RU2AS, hsp70-2, ARTC1, B-RAF, beta-catenin, CDC27, CDK4, CDK12, CDKN2A, CLPP, CSNK1A1, FN1, GAS7, GPNMB, HAUS3, LDLR-fucosyltransferase, MART2, MATN, MUM1, MUM2, MUM3, neo-PAP, myosin, PPP1R3B, PRDX5, PTPRK, RBAF600, SIRT2, SNRPD1, trioephosphate isomerase, OA1, RAB38, TRP1, TRP2, melan-A, BAGE1, GAGE1, GAGE2, GAGE8, GAGE3, GAGE4, GAGE5, GAGE6, GAGE7, GNTVF, LY6K, TRAG3, CASP8, SAGE, DEK-CAN, EFTUD2, FLT3-ITD, cyclin A1, FNDC3B, MAGEAG, G250, hepsin, intestinal carboxyl esterase, PBF, CASP5, COA1, OGT, OS9, CALCA, MDM2, alpha actinin4, elongation factor 2, fos-related antigen 1, legumain, sperm protein 17, carbonic anhydrase IX, folate receptor-α, neutrophil elastase, ephrinB2, glioma-associated antigen, β-human chorionic gonadotropin, alphafetoprotein thyroglobulin, telomerase reverse transcriptase, intestinal carboxy esterase, prostein, and survivin.

Recombinant microorganisms described herein can be produced by standard molecular biology and microbiology techniques known to the skilled artisan. For example, recombinant viruses described herein can be propagated in suitable host cells, e.g., HeLa cells, 293 cells, or Vero cells, isolated from host cells and stored in conditions that promote stability and integrity of the virus, such that loss of infectivity over time is minimized. In some embodiments, recombinant viruses described herein can be propagated in host cells using cell stacks, roller bottles, or perfusion bioreactors. In some examples, downstream methods for purification of the recombinant oncolytic viruses can comprise filtration (e.g., depth filtration, tangential flow filtration, or a combination thereof), ultracentrifugation, or chromatographic capture. In some embodiments, the recombinant virus can be stored, e.g., by freezing or drying, such as by lyophilization. In some embodiments, prior to administration, the stored recombinant virus can be reconstituted (if dried for storage) and diluted in a pharmaceutically acceptable carrier for administration.

### III. Methods of Treating Cancer

Methods provided herein include methods of treating cancer in a subject. The terms “cancer” and “tumor” are used interchangeably herein, wherein the term “cancer” as used herein refers to hyperproliferative conditions. Cancers that are treated by compositions and method comprised herein can include solid cancers or liquid cancers. Cancers that are treated by methods described herein include, but are not limited to, melanoma, hepatocellular carcinoma, breast cancer, lung cancer, peritoneal cancer, prostate cancer, bladder cancer, ovarian cancer, leukemia, lymphoma, renal carcinoma, renal adenocarcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma.

Cancer cells that are treated by the methods of this disclosure include cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some cases, solid cancers that are metastatic are treated using methods provided herein. In some cases, solid cancers that are inaccessible or difficult to access, such as for purpose of intratumoral delivery of therapeutic agents, are treated using the methods described herein. Cancers that are associated with increased expression of free fatty acids can, in some examples, may be treated using methods described herein.

Methods provided herein include methods of inhibiting or preventing local invasiveness or metastasis, or both, of any type of primary cancer. For example, the primary cancer can be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In some embodiments, the primary cancer is lung cancer, e.g., non-small cell lung carcinoma.

Methods provided herein include methods of preventing cancer and methods of treating pre-cancers or premalignant cells, including metaplasias, dysplasias, and hyperplasias. Method provided herein further include, methods of inhibiting undesirable but benign cells, such as squamous metaplasia, dysplasia, benign prostate hyperplasia cells, hyperplastic lesions, and the like. In some embodiments, the progression to cancer or to a more severe form of cancer is halted, disrupted, or delayed by methods provided herein.

In some embodiments, treatment of a cancer can be detected by one or more of a decrease in the volume of the tumor in the subject, a decrease in the level of expression of one or more tumor marker in the subject or a sample from the subject, or a decrease in the number of tumor sites in the subject. Methods of evaluating the volume and metastasis of solid tumors in humans are known in the art. These can include, for example, positron emission tomography (PET) scanning, magnetic resonance imaging (MRI), ultrasound, CT angiography, and X-ray. Tumor proteins can be detected in vivo or in an ex vivo sample from a subject. In some embodiments, the sample from the subject can be a blood, tissue, urine, bone marrow, tumor biopsy, or saliva sample. Tumor proteins can be detected through standard assays known to the skilled artisan including for example, but not limiting to, standard immunoassays, such as ELISA, flow cytometry, immunohistochemistry, and RIA.

### IV. Methods of Enhancing an Immune Response

Methods provided herein include methods of inducing or enhancing an immune response in vivo in a subject. In some embodiments the immune response induced or enhanced is directed to one or more cancer associated antigens released from one or more cancer cell lysed in vivo by a recombinant microorganism described herein.

In some embodiments, the immune response can be a cell-mediated or humoral response. In some embodiments, the immune response can be detected by assaying for an enhancement of B-cell proliferation, CD3+ T cell proliferation, CD4+ T cell proliferation, CD8+ T cell proliferation, or any combinations thereof. In some embodiments, the immune response can be detected by assaying for an enhancement of production of: IL-2, IFN-γ, IL-1, IL-4, IL-5, IL-6, IL-13, IL-17, IL-21, IL-22, TNFα, CSF, TGFβ, granzyme, and the like. In some embodiments, cytokine release may be quantified using ELISA, flow cytometry, western blot, or any combinations thereof. In some embodiments, the immune response can be detected by an enhancement of antigen presenting cell proliferation, function or any combinations thereof. In some embodiments, the immune response can be detected by one or more of a decrease in the volume of the tumor in the subject, stabilization of a tumor, a decrease in the level of expression of one or more tumor markers in the subject or a sample from the subject, or a decrease in the number of tumor sites in the subject. Methods of evaluating the volume and metastasis of solid tumors in humans are known in the art. These can include, for example, positron emission tomography (PET) scanning, magnetic resonance imaging (MM), ultrasound, CT angiography, and X-ray.

Tumor proteins can be detected in vivo or in an ex vivo sample from a subject. In some embodiments, the sample from the subject can be a blood, tissue, urine, bone marrow or saliva sample. Tumor proteins can be detected through standard assays known to the skilled artisan including for example, but not limiting to, standard immunoassays, such as ELISA, immunohistochemistry, flow cytometry, and RIA.

There are an additional variety of in vitro and in vivo assays are known in the art for measuring an immune response, including measuring humoral and cellular immune responses, which include but are not limited to standard immunoassays, such as RIA, ELISA assays, immunohistochemistry, intracellular staining (such as intracellular cytokine staining, See, e.g., Smith et al., PLoS One (2015) 10(9): e0138042); T cell assays including for example, lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Recent methods of measuring cell-mediated immune response include measurement of intracellular cytokines or cytokine secretion by T-cell populations, or by measurement of epitope specific T-cells (e.g., by the tetramer technique) (reviewed by McMichael, A. J., and O'Callaghan, C. A., J. Exp. Med. 187(9)1367-1371, 1998; Mcheyzer-Williams, M. G., et al., Immunol. Rev. 150:5-21, 1996; Lalvani, A., et al., J. Exp. Med. 186:859-865, 1997). In illustrative embodiments disclosed herein, the enzyme-linked immunospot (ELISPOT) assay is used to detect and analyze individual cells that secrete interferon-γ (IFN-γ). ELISPOT IFN-γ assays and reagents are available at BD Biosciences 2350 Qume Drive San Jose, Calif., 95131. The ELISPOT assay is capable of detecting cytokine producing cells from both activated naïve and memory T-cell populations and derives its specificity and sensitivity by employing high affinity capture and detection antibodies and enzyme-amplification. Additional information regarding the use of ELISPOT assay is provided in J. Immunol. Methods. 2001, 254(1-2):59. Animal models, e.g. non-human primates, are known in the art. For example, the mouse is an accepted model for human immune response. Mouse NK cell response to tumors is an accepted model for human NK cell response to tumors. Additionally, mouse T cells are a model for human T cells, mouse dendritic cells (DCs) are a model for human DCs, mouse NKT cells are a model for human NKT cells, mouse innate response is an accepted model for human innate response, and so on. Model studies are disclosed, for example, for CD8⁺ T cells, central memory T cells, and effector memory T cells (see, e.g., Walzer, et al. (2002) J. Immunol. 168:2704-2711); the two subsets of NK cells (see, e.g., Chakir, et al. (2000) J. Immunol. 165:4985-4993; Smith, et al. (2000) J. Exp. Med. 191:1341-1354; Ehrlich, et al. (2005) J. Immunol. 174:1922-1931; Peritt, et al. (1998) J. Immunol. 161:5821-5824); NKT cells (see, e.g., Couedel, et al. (1998) Eur. J. Immunol. 28:4391-4397; Sakamoto, et al. (1999) J. Allergy Clin. Immunol. 103:S445-S451; Saikh, et al. (2003) J. Infect. Dis. 188:1562-1570; Emoto, et al. (1997) Infection Immunity 65:5003-5009; Taniguchi, et al. (2003) Annu. Rev. Immunol. 21:483-513; Sidobre, et al. (2004) Proc. Natl. Acad. Sci. 101:12254-12259); monocytes/macrophages (Sunderkotter, et al. (2004) J. Immunol. 172:4410-4417); the two lineages of DCs (Boonstra, et al. (2003) J. Exp. Med. 197:101-109; Donnenberg, et al. (2001) Transplantation 72:1946-1951; Becker (2003) Virus Genes 26:119-130; Carine, et al. (2003) J. Immunol. 171:6466-6477; Penna, et al. (2002) J. Immunol. 69:6673-6676; Alferink, et al. (2003) J. Exp. Med. 197:585-599). Mouse innate response, including the Toll-Like Receptors (TLRs), is a model for human innate immune response, as disclosed (see, e.g., Janssens and Beyaert (2003) Clinical Microb. Revs. 16:637-646). Mouse neutrophils are an accepted model for human neutrophils (see, e.g., Kobayashi, et al. (2003) Proc. Natl. Acad. Sci. USA 100:10948-10953; Torres, et al. (2004) 72:2131-2139; Sibelius, et al. (1999) Infection Immunity 67:1125-1130; Tvinnereim, et al. (2004) J. Immunol. 173:1994-2002). Murine immune response to Listeria is an accepted model for human response to Listeria (see, e.g., Kolb-Maurer, et al. (2000) Infection Immunity 68:3680-3688; Brzoza, et al. (2004) J. Immunol. 173:2641-2651).

In some embodiments, the immune response may be measured by one or more of intracellular cytokine staining (ICS), ELISpot, proliferation assays, cytotoxic T-cell assays including chromium release or equivalent assays, and gene expression analysis using any number of polymerase chain reaction (PCR) or RT-PCR based assays, as described herein as well as any other suitable assays for measuring immune response. In some cases, samples of cellular products or apheresis products can be cryopreserved for retrospective analysis of cell phenotype and function.

### V. Administration

Methods described herein include administering at least two recombinant microorganisms (e.g., recombinant viruses) described herein in a prime-boost regimen. Prime-boost regimens are known to the skilled artisan and known in the art. Prime-boost regimens are generally characterized by administration of a first priming administration and one or more subsequent boosting administrations.

In some embodiments, the prime-boost regimen can be heterologous. A heterologous prime-boost regimen as described herein can comprise the use of different means for priming and for boosting the immune response. A heterologous prime-boosting regimen as described herein can comprise administering a first recombinant microorganism (prime) and subsequently administering a second recombinant microorganism (boost), wherein the first and second recombinant microorganisms are different.

In some embodiments, the first (prime) recombinant microorganism can be a virus and the second (boost) recombinant microorganism can be a virus, wherein the first (prime) and second (boost) viruses are different. In some embodiments, the first (prime) and second (boost) viruses can be serologically distinct. In some embodiments, the first (prime) and second (boost) viruses can be classified in different taxonomic families. In some embodiments, the first (prime) and second (boost) viruses can be classified in different taxonomic species. In some embodiments, the first (prime) recombinant virus can be a virus described herein, and the second (boost) recombinant virus can be a different virus described herein.

In some embodiments, the first (prime) recombinant microorganism can be a bacterium and the second (boost) recombinant microorganism can be a bacterium, wherein the first (prime) and second (boost) bacteria are different. In some embodiments, the first (prime) and second (boost) bacteria can be serologically distinct. In some embodiments, the first (prime) and second (boost) bacteria can be classified in different taxonomic families. In some embodiments, the first (prime) and second (boost) bacteria can be classified in different taxonomic species. In some embodiments, the first (prime) recombinant bacteria can be a bacterium described herein, and the second (boost) recombinant bacteria can be a different virus described herein.

In some embodiments, the first (prime) recombinant microorganism can be a parasite and the second (boost) recombinant microorganism can be a parasite, wherein the first (prime) and second (boost) parasites are different. In some embodiments, the first (prime) and second (boost) parasites can be serologically distinct. In some embodiments, the first (prime) and second (boost) parasites can be classified in different taxonomic families. In some embodiments, the first (prime) and second (boost) parasites can be classified in different taxonomic species. In some embodiments, the first (prime) recombinant parasites can be a virus described herein, and the second (boost) recombinant virus can be a different parasite described herein.

In some embodiments, the first (prime) recombinant microorganism can be a virus and the second (boost) recombinant microorganism can be a bacterium. In some embodiments, the first (prime) recombinant virus can be a virus described herein, and the second (boost) recombinant bacterium can be a bacterium described herein. In some embodiments, the first (prime) recombinant microorganism can be a bacterium and the second (boost) recombinant microorganism can be a virus. In some embodiments, the first (prime) recombinant bacterium can be a bacterium described herein, and the second (boost) recombinant virus can be a virus described herein.

In some embodiments, the first (prime) recombinant microorganism can be a virus and the second (boost) recombinant microorganism can be a parasite. In some embodiments, the first (prime) recombinant virus can be a virus described herein, and the second (boost) recombinant parasite can be a parasite described herein. In some embodiments, the first (prime) recombinant microorganism can be a parasite and the second (boost) recombinant microorganism can be a virus. In some embodiments, the first (prime) recombinant parasite can be a parasite described herein, and the second (boost) recombinant virus can be a virus described herein.

In some embodiments, the first (prime) recombinant microorganism can be a bacterium and the second (boost) recombinant microorganism can be a parasite. In some embodiments, the first (prime) recombinant bacterium can be a bacterium described herein, and the second (boost) recombinant parasite can be a parasite described herein. In some embodiments, the first (prime) recombinant microorganism can be a parasite and the second (boost) recombinant microorganism can be a bacterium. In some embodiments, the first (prime) recombinant parasite can be a parasite described herein, and the second (boost) recombinant bacterium can be a bacterium described herein.

In some embodiments, the prime-boost regimen can comprise administering a single prime recombinant microorganism and one or more (e.g., 2, 3, 4, 5, or more) subsequent boosting recombinant microorganisms. In some embodiments, the heterologous prime-boost regimen can comprise administering a single prime recombinant microorganism, and two or more (e.g., 3, 4, 5, or more) subsequent boosting recombinant microorganisms, wherein the two or more subsequent boosting recombinant microorganisms are different from each other. In some embodiments, the heterologous prime-boost regimen can comprise administering a single prime recombinant microorganism, and two or more (e.g., 3, 4, 5, or more) subsequent boosting recombinant microorganisms, wherein the two subsequent boosting recombinant microorganisms are the same as each other.

In some embodiments, the one or more boosting administrations can be administered at intervals comprising days, weeks or months after administration of the initial priming administration. In some embodiments, the one or more boosting administrations are administered at intervals of 1, 2, 3, 4, 5, 6, 7 or more days after administration of the initial priming administration. In some embodiments, the one or more boosting administrations can be administered at intervals of 1, 2, 3, 4, 5, 6, 7, 8 or more weeks after administration of the initial priming administration. In some embodiments, the one or more boosting administrations can be administered at intervals of 1, 2, 3, 4, 5, 6, 7, 8, 9, 10, 11, 12 or more months after administration of the initial priming administration. In some embodiments, the one or more boosting administrations can be administered at any combination of intervals after administration of the initial the priming administrations) (e.g., 1, 2, 3, 4, 5, 6, 7 or more days, 1, 2, 3, 4, 5, 6, 7, 8 or more weeks, or 1, 2, 3, 4, 5, 6, 7, 8, 9, 10, 11, 12 or more months).

In some embodiments, a first boost can be administered within 1, 2, 3, 4, 5, 6, 7 days or weeks of the prime administration. In some embodiments, a second boost can be administered within 1, 2, 3, 4, 5, 6, 7 days or weeks of the first boost administration.

In embodiments, the one or more boost administrations can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

The timing of the prime and boost administration can be determined by the skilled artisan. The administration of one or more than one boost administration can be determined by the skilled artisan. In some embodiments, the prime dose administered can be lower than the boost dose administered. In some embodiments, the prime dose administered can be higher than the boost dose administered. The dose of the prime and boost administered can be determined by the skilled artisan.

In some embodiments, the prime dose can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In some embodiments, the prime dose can refer to the amount administered to a subject or a tumor over a 1, 2, 5, 10, 15, 20, or 24-hour period.

In some embodiments, the boost dose can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In some embodiments, the boost dose can refer to the amount administered to a subject or a tumor over a 1, 2, 5, 10, 15, 20, or 24-hour period.

In some embodiments, a dose of a first (prime) recombinant microorganism described herein and a dose of a second (boost) recombinant microorganism described herein can be administered to a subject. The amount of the prime and the boost dosage of the recombinant microorganisms can be determined by the skilled artisan.

In some embodiments, a dose of a first (prime) recombinant microorganism is auxotrophic (aroA mutant) Salmonella Typhimurium strains and a dose of a second (boost) recombinant microorganism is vaccinia virus (WR.TK−). In some embodiments, a dose of a first (prime) recombinant microorganism is from a vaccinia virus and a dose of a second (boost) recombinant microorganism is from at least one of a Herpes simplex virus strain or Vesicular stomatitis virus (VSV). In an aspect, a Herpes simplex virus can be modified. Modifications can include additions, insertions, truncations, mutations, and any combination thereof. In an aspect, a gene involved in a neurovirulence, pathogenesis, or replication can be modified. In some embodiments an HSV modification can be any one of a gamma 34.5 or ICP6 deletion.

In some embodiments, the prime recombinant microorganism can be a recombinant virus and the boost recombinant microorganism can be a different recombinant virus, and the amount of the prime and/or boost that is administered can be from about 10³ to 10¹² infectious viral particles or plaque forming units (PFU), or from about 10⁵ to 10¹⁰ PFU, or from about 10⁵ to 10⁸ PFU, or from about 10⁸ to 10¹⁰ PFU. In some embodiments, the amount of a recombinant virus of this disclosure administered to a subject can be from about 10³ to 10¹² viral particles or plaque forming units (PFU), or from about 10⁵ to 10¹⁰ PFU, or from about 10⁵ to 10⁸ PFU, or from about 10⁸ to 10¹⁰ PFU. In some embodiments, a recombinant virus of this disclosure can be administered at a dose that comprises from about 10³ PFU/dose to about 10⁴PFU/dose, from about 10⁴ PFU/dose to about 10⁵ PFU/dose, from about 10⁵ PFU/dose to about 10⁶ PFU/dose, from about 10⁷ PFU/dose to about 10⁸ PFU/dose, from about 10⁹ PFU/dose to about 10¹⁰ PFU/dose, from about 10¹⁰ PFU/dose to about 10¹¹PFU/dose, from about 10¹¹ PFU/dose to about 10¹²PFU/dose, from about 10¹²PFU/dose to about 10¹³ PFU/dose, from about 10¹³ PFU/dose to about 10¹⁴ PFU/dose, or from about 10¹⁴ PFU/dose to about 10¹⁵ PFU/dose. In some embodiments, a recombinant virus of this disclosure can be administered at a dose that comprises about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴PFU/dose, about 3×10⁴PFU/dose, about 4×10⁴PFU/dose, about 5×10⁴PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose, 5×10⁵PFU/dose, 6×10⁵ PFU/dose, 7×10⁵PFU/dose, 8×10⁵PFU/dose, 9×10⁵PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶PFU/dose, about 8×10⁶PFU/dose, about 9×10⁶PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸PFU/dose, about 2×10⁸PFU/dose, about 3×10⁸PFU/dose, about 4×10⁸PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹PFU/dose, about 2×10¹¹PFU/dose, about 3×10¹¹PFU/dose, about 4×10¹¹PFU/dose, about 5×10¹¹PFU/dose, about 6×10¹¹PFU/dose, about 7×10¹¹PFU/dose, about 8×10¹¹PFU/dose, about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose.

In some embodiments, a recombinant virus of this disclosure can be administered at a dose that comprises from about 10³ viral particles/dose to about 10⁴ viral particles/dose, from about 10⁴ viral particles/dose to about 10⁵ viral particles/dose, from about 10⁵ viral particles/dose to about 10⁶ viral particles/dose, from about 10⁷ viral particles/dose to about 10⁸ viral particles/dose, from about 10⁹ viral particles/dose to about 10¹⁰ viral particles/dose, from about 10¹⁰ viral particles/dose to about 10¹¹ viral particles/dose, from about 10¹¹ viral particles/dose to about 10¹² viral particles/dose, from about 10¹² viral particles/dose to about 10¹³ viral particles/dose, from about 10¹³ viral particles/dose to about 10¹⁴ viral particles/dose, or from about 10¹⁴ viral particles/dose to about 10¹⁵ viral particles/dose.

In some embodiments, a recombinant virus of this disclosure can be administered at a dose that comprises from about 10³ PFU/kg to about 10⁴ PFU/kg, from about 10⁴ PFU/kg to about 10⁵ PFU/kg, from about 10⁵ PFU/kg to about 10⁶ PFU/kg, from about 10⁷ PFU/kg to about 10⁸ PFU/kg, from about 10⁹ PFU/kg to about 10¹⁰ PFU/kg, from about 10¹⁰ PFU/kg to about 10¹¹ PFU/kg, from about 10¹¹ PFU/kg to about 10¹² PFU/kg, from about 10¹² PFU/kg to about 10¹³ PFU/kg, from about 10¹³ PFU/kg to about 10¹⁴ PFU/kg, or from about 10¹⁴ PFU/kg to about 10¹⁵ PFU/kg. In some embodiments, a modified oncolytic virus of this disclosure, such as an oncolytic vaccinia virus, is administered at a dose that comprises about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴PFU/kg, about 2×10⁴PFU/kg, about 3×10⁴PFU/kg, about 4×10⁴PFU/kg, about 5×10⁴PFU/kg, about 6×10⁴PFU/kg, about 7×10⁴PFU/kg, about 8×10⁴PFU/kg, about 9×10⁴PFU/kg, about 10⁵PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵PFU/kg, 9×10⁵PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹PFU/kg, about 2×10¹¹PFU/kg, about 3×10¹¹PFU/kg, about 4×10¹¹PFU/kg, about 5×10¹¹PFU/kg, about 6×10¹¹ PFU/kg, about 7×10¹¹PFU/kg, about 8×10¹¹PFU/kg, about 9×10¹¹PFU/kg, or about 10¹²PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure, such as an oncolytic vaccinia virus, is administered at a dose that comprises 5×10⁹ PFU/kg. In some embodiments, a modified oncolytic virus of this disclosure, such as an oncolytic vaccinia virus, is administered at a dose that comprises up to 5×10⁹ PFU/kg.

In some embodiments, a recombinant virus of this disclosure can be administered at a dose that comprises from about 10³ viral particles/kg to about 10⁴ viral particles/kg, from about 10⁴ viral particles/kg to about 10⁵ viral particles/kg, from about 10⁵ viral particles/kg to about 10⁶ viral particles/kg, from about 10⁷ viral particles/kg to about 10⁸ viral particles/kg, from about 10⁹ viral particles/kg to about 10¹⁰ viral particles/kg, from about 10¹⁰ viral particles/kg to about 10¹¹ viral particles/kg, from about 10¹¹ viral particles/kg to about 10¹² viral particles/kg, from about 10¹² viral particles/kg to about 10¹³ viral particles/kg, from about 10¹³ viral particles/kg to about 10¹⁴ viral particles/kg, or from about 10¹⁴ viral particles/kg to about 10¹⁵ viral particles/kg.

A liquid dosage form of a recombinant virus as described herein can comprise, in some embodiments, a viral dose from about 10³ PFU/mL to about 10⁴ PFU/mL, from about 10⁴ PFU/mL to about 10⁵ PFU/mL, from about 10⁵ PFU/mL to about 10⁶ PFU/mL, from about 10⁷ PFU/mL to about 10⁸PFU/mL, from about 10⁹ PFU/mL to about 10¹⁰ PFU/mL, from about 10¹⁰ PFU/mL to about 10¹¹PFU/mL, from about 10¹¹ PFU/mL to about 10¹²PFU/mL, from about 10¹²PFU/mL to about 10¹³ PFU/mL, from about 10¹³ PFU/mL to about 10¹⁴ PFU/mL, or from about 10¹⁴ PFU/mL to about 10¹⁵ PFU/mL.

In some embodiments, a recombinant virus of this disclosure can be administered at a dose that comprises about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴PFU/mL, about 8×10⁴PFU/mL, about 9×10⁴PFU/mL, about 10⁵PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸PFU/mL, about 8×10⁸PFU/mL, about 9×10⁸PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹PFU/mL, about 9×10¹¹PFU/mL, or about 10¹²PFU/mL, about 10¹²PFU/mL to about 10¹³ PFU/mL, about 10¹³ PFU/mL to about 10¹⁴ PFU/mL, or about 10¹⁴ PFU/mL to about 10¹⁵ PFU/mL.

In some embodiments, where the recombinant virus is administered by an injection. In some embodiments, the dosage can comprise about 10³ viral particles per injection, 10⁴ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In some embodiments, the dosage can comprise about 10³ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. In additional embodiments, a recombinant virus of this disclosure can be administered at a dose that is about 10³ Tissue Culture Inhibitor Dose 50% (TCID₅₀)/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 3×10⁸ TCID₅₀/kg, 4×10⁸ TCID₅₀/kg, 5×10⁸ TCID₅₀/kg, 3×10⁹ TCID₅₀/kg, 4×10⁹ TCID₅₀/kg, 5×10⁹ TCID₅₀/kg, 3×10¹⁰ TCID₅₀/kg, 4×10¹⁰ TCID₅₀/kg, or 4×10¹⁰ TCID₅₀/kg.

The routes of administration can vary with the location and nature of the tumor. In certain embodiments, the route of administration is transdermal, parenteral, intramuscular, subcutaneous, regional (e.g., in the proximity of a tumor, particularly with the vasculature or adjacent vasculature of a tumor), percutaneous, intrathecal, intranasal, intratracheal, intraperitoneal, intraarterial, intravesical, intratumoral, inhalation, perfusion, by lavage or orally.

In some embodiments, an injectable dose of the pharmaceutical composition can be administered as a bolus injection or as a slow infusion. In some embodiments, the pharmaceutical composition can be administered to the subject from a source implanted in the subject. In certain embodiments, administration of the pharmaceutical composition can occur by continuous infusion over a selected period of time. In some embodiments, the pharmaceutical composition can be administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. In some embodiments, the pharmaceutical composition can be administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

In some embodiments, the methods can comprise administering a prime-boost regimen as disclosed herein, followed by an administration of a further recombinant microorganism, such as a third recombinant microorganism, a fourth recombinant microorganism, or any combinations thereof. In some embodiments, the methods can comprise administering a prime-boost regimen as disclosed herein, followed by, and preceded by or in combination with one or more further therapy. Examples of the further therapy can include, but are not limited to, chemotherapy, radiation, oncolytic viral therapy with an additional virus, treatment with immunomodulatory proteins, an anti-cancer agent, or any combinations thereof. The further therapy can be administered concurrently or sequentially with respect to administration of the prime, boost, or prime-boost administrations. In some embodiments, the methods can comprise administering a prime, boost, or prime-boost regimen as disclosed herein, followed by, preceded by, or in combination with one or more anti-cancer agents or cancer therapies. Anti-cancer agents can include, but are not limited to, chemotherapeutic agents, radiotherapeutic agents, cytokines, immune checkpoint inhibitors, anti-angiogenic agents, apoptosis-inducing agents, anti-cancer antibodies and anti-cyclin-dependent kinase agents. In some embodiments, the cancer therapies include chemotherapy, biological therapy, radiotherapy, immunotherapy, hormone therapy, anti-vascular therapy, cryotherapy, toxin therapy, surgery or combinations thereof.

In certain embodiments, methods can comprise administering a prime-boost regimen as described herein, in combination with one or immunomodulatory agents. Immunomodulatory agents can include any compound, molecule or substance capable of suppressing anti-recombinant microorganism immunity (e.g., anti-viral immunity) associated with a tumor or cancer. In some embodiments, the immunomodulatory agent can be capable of suppressing innate immunity or adaptive immunity to the recombinant microorganism (e.g., recombinant virus). Non-limiting examples of immunomodulatory agents include anti-CD33 antibody or variable region thereof, an anti-CD11b antibody or variable region thereof, a COX2 inhibitor, e.g., celecoxib, cytokines, such as IL-12, GM-CSF, IL-2, IFN3 and 1FNγ, and chemokines, such as MIP-1, MCP-1 and IL-8. In some embodiments, the immunomodulatory agent can include immune checkpoint modulators such as, but not limited to, anti-CTLA4, anti-PD-1, and anti-PD-L1 and TLR agonists (e.g., Poly I:C). In some examples, the immunomodulatory agent can include an immune checkpoint inhibitor, such as an antagonist of PD-1 (e.g., an antagonist antibody that binds to PD-1), an antagonist of PD-L1 (e.g., an antagonist antibody that binds to PD-L1), an antagonist of CTLA-4 (e.g., an antagonist antibody that binds to CTLA-4), an antagonist of A2AR (e.g., an antagonist antibody that binds to A2AR), an antagonist of B7-H3 (e.g., an antagonist antibody that binds to B7-H3), an antagonist of B7-H4 (e.g., an antagonist antibody that binds to B7-H4), an antagonist of BTLA (e.g., an antagonist antibody that binds to BTLA), an antagonist of IDO (e.g., an antagonist antibody that binds to IDO), an antagonist of KIR (e.g., an antagonist antibody that binds to KIR), an antagonist of LAG3 (e.g., an antagonist antibody that binds to LAG3), an antagonist of TIM-3 (e.g., an antagonist antibody that binds to TIM3). In some embodiments, the immune checkpoint inhibitor can comprise a protein that binds to PD-1, PD-L1, CTLA-4, A2AR, B7-H3, B7-H4, BTLA, IDO, KIR, LAG3, TIM-3, VISTA, CD160, TIGIT, PSGL-1, or any combinations thereof.

In some embodiments, the further therapy can comprise administering an immune checkpoint regulator. In some embodiments, the immune checkpoint regulator can be TGN1412. In some embodiments, the immune checkpoint regulator can be NKTR-214. In some embodiments, the immune checkpoint regulator can be MEDI0562. In some embodiments, the immune checkpoint regulator can be MEDI6469. In some embodiments, the immune checkpoint regulator can be MEDI6383. In some embodiments, the immune checkpoint regulator can be JTX-2011. In some embodiments, the immune checkpoint regulator can be Keytruda (pembrolizumab). In some embodiments, the immune checkpoint regulator can be Opdivo (nivolumab). In some embodiments, the immune checkpoint regulator can be Yervoy (ipilimumab). In some embodiments, the immune checkpoint regulator can be tremelimumab. In some embodiments, the immune checkpoint regulator can be Tecentriq (atezolizumab). In some embodiments, the immune checkpoint regulator can be MGA271. In some embodiments, the immune checkpoint regulator can be indoximod. In some embodiments, the immune checkpoint regulator can be Epacadostat. In some embodiments, the immune checkpoint regulator can be lirilumab. In some embodiments, the immune checkpoint regulator can be BMS-986016. In some embodiments, the immune checkpoint regulator can be MPDL3280A. In some embodiments, the immune checkpoint regulator can be avelumab. In some embodiments, the immune checkpoint regulator can be durvalumab. In some embodiments, the immune checkpoint regulator can be MEDI4736. In some embodiments, the immune checkpoint regulator can be MEDI4737. In some embodiments, the immune checkpoint regulator can be TRX518. In some embodiments, the immune checkpoint regulator can be MK-4166. In some embodiments, the immune checkpoint regulator can be urelumab (BMS-663513). In some embodiments, the immune checkpoint regulator can be PF-05082566 (PF-2566)

In some embodiments the further therapy can be radiation. Exemplary doses include, but are not limited to, 5,000 Rads (50 Gy) to 100,000 Rads (1000 Gy), or 50,000 Rads (500 Gy), or other appropriate doses within the recited ranges. In some embodiments, the radiation dose can be about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In some embodiments, the further therapy can be chemotherapy. Exemplary chemotherapeutic agents can include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

Exemplary alkylating agents can include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines include, without limitation, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids can include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteasome inhibitors can include, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

The further therapy can be administered, in various embodiments, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain embodiments, the further therapy can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the further therapy can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

In some embodiments, further therapy can comprise vaccines, colony stimulating agents, interferons, interleukins, viruses, anti-angiogenic agents, antigens, co-stimulatory agents, immunogenicity agents, immunomodulators, or immunotherapeutic agents.

### VI. Pharmaceutical Compositions

Compositions described herein include, pharmaceutical compositions containing one or more recombinant microorganism described herein. In some embodiments, the pharmaceutical compositions herein can be in unit dose form. Pharmaceutical compositions can be prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, the pharmaceutical composition comprises a solubilizer, such as sterile water, Tris-buffer. In some embodiments, the pharmaceutical composition comprises an excipient. Exemplary excipients are known in the art, e.g., as described in the Handbook of Pharmaceutical Excipients, American Pharmaceutical Association (1986). Example of excipients include, but are not limited to, a buffering agent, a preservative, a stabilizer, a binder, a compaction agent, a lubricant, a chelator, a dispersion enhancer, a disintegration agent, a flavoring agent, a sweetener, and a coloring agent.

In some embodiments, the excipient can be a buffering agent. Non-limiting examples of buffering agents can include sodium citrate, magnesium carbonate, magnesium bicarbonate, calcium carbonate, and calcium bicarbonate. As buffering agents, sodium bicarbonate, potassium bicarbonate, magnesium hydroxide, magnesium lactate, magnesium gluconate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some embodiments, the excipient can comprise a preservative. Non-limiting examples of preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments, the pharmaceutical composition can comprise a binder as an excipient. Non-limiting examples of binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. The binders that are used in a pharmaceutical formulation is selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, the pharmaceutical composition can comprise a lubricant as an excipient. Non-limiting examples of lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, the pharmaceutical formulation can comprise a dispersion enhancer as an excipient. Non-limiting examples of dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the pharmaceutical composition can comprise a disintegrant as an excipient. In some embodiments a disintegrant can be a non-effervescent disintegrant. Non-limiting examples of non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments, the pharmaceutical composition can comprise a chelator. In some embodiments, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

Also contemplated herein are combination products that can include one or more recombinant microorganism disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nystatin, amorolfine, butenafine, naftifine, terbinafine, and other topical agents. In some embodiments, the pharmaceutical composition comprises an additional agent. In some embodiments, an additional agent can be present in a therapeutically effective amount in the pharmaceutical composition.

In some embodiments, the pharmaceutical compositions described herein can comprise a preservative, for example to prevent the growth of microorganisms. In some embodiments, the pharmaceutical compositions can comprise a preservative. In some embodiments, the pharmaceutical compositions do not comprise a preservative. The pharmaceutical forms suitable for injectable use include sterile aqueous solutions or dispersions and sterile powders for the extemporaneous preparation of sterile injectable solutions or dispersions. The pharmaceutical compositions can comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form can be suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage may be dissolved in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions can be prepared by incorporating a modified oncolytic virus according to the present disclosure, such as oncolytic vaccinia viruses as described herein or a pharmaceutical composition containing the same, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions can be administered in a manner compatible with the dosage formulation and in such amount as is therapeutically effective.

In certain embodiments, the pharmaceutical composition of this disclosure can comprise an effective amount of a modified virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers can include phosphate buffered saline solutions, water, emulsions, such as oil/water emulsions, various types of wetting agents and sterile solutions. Additional non-limiting examples of pharmaceutically compatible carriers include gels, bioadsorbable matrix materials, implantation elements containing the modified oncolytic virus or any other suitable vehicle, delivery or dispensing means or material. Such carriers can be formulated by conventional methods and administered to the subject at an effective amount.

### VII. Kits

Provided herein are kits comprising a prime composition, one or more boost composition, or a prime compositions and one or more boost composition described herein. In some embodiments, the kit can comprise a recombinant microorganism composition for prime administration. In some embodiments, the kit can comprise one or more recombinant microorganism compositions for one or more boost administration. In some embodiments, the kit can comprise a recombinant microorganism composition for prime administration and one or more recombinant microorganism compositions for one or more boost administration.

In certain embodiments, the kit can comprise one or more containers containing a recombinant microorganism composition for prime administration. In certain embodiments, the kit can comprise one or more containers containing a recombinant microorganism composition for boost administration. In certain embodiments, the kit can comprise one or more containers containing a recombinant microorganism composition for prime administration and one or more containers containing a recombinant microorganism composition for one or more boost administration.

In some embodiments, the kit includes instructions for use, a device for administering the recombinant microorganism composition to a subject, or a device for administering an additional agent or compound to a subject. In some embodiments, the instructions can comprise a description of the modified recombinant microorganism and, optionally, other components included in the kit, and methods for administration, including methods for determining the proper state of the subject, the proper dosage amount and the proper administration method for administering the recombinant microorganism. Instructions can also include guidance for monitoring the subject over duration of the treatment time. In some embodiments, the kit can comprise one or more agents, e.g., at least one of an anti-cancer agent, an immunomodulatory agent, or any combinations thereof, that are administered in combination with the recombinant microorganism composition described herein.

In some embodiments, the kit includes a device for administering the recombinant microorganism composition to a subject. Any of a variety of devices known in the art for administering medications and pharmaceutical compositions is included in the kits provided herein. For example, and not by way of limitation, such devices include, a hypodermic needle, an intravenous needle, a catheter, a needle-less injection device, an inhaler and a liquid dispenser, such as an eyedropper. In certain embodiments, a recombinant microorganism to be delivered systemically, for example, by intravenous injection, an intratumoral injection, an intraperitoneal injection, are included in a kit with a hypodermic needle and syringe.

### EXAMPLES

The examples below further illustrate the described embodiments without limiting the scope of this disclosure.

### Example 1: Heterologous Prime-Boost Regimen of Salmonella Bacterium or Vaccinia Virus in a Mouse Renal Adenocarcinoma Tumor Model

A heterologous prime boost regimen was studied using BALB/c mice with RENCA tumors as follows. The regimen used the tumor selective strains of Vaccinia virus with the TK deletion and the attenuated auxotrophic (aroA mutant) Salmonella Typhimurium strains, which were given by an intratumoral injection to mice at suboptimal doses. Mice were injected subcutaneously with 1×10⁵ RENCA cells and treatment began once tumors reached 50-100 mm³. Mice were assigned to one of the four groups shown in Table 1.

TABLE 1
Treatment groups of RENCA-tumor mice dosed with
salmonella bacterium or Vaccinia virus

| Group | Group name | Prime-Boost Regimen |
|---|---|---|
| 1 | Control (Vehicle) | — |
| 2 | WR.Tk− | Vaccinia (day 1) |
| 3 | Salmonella + TK− | Salmonella (day 1); Vaccinia (day 7) |
| 4 | TK− + Salmonella | Vaccinia (day 1); Salmonella (day 7) |

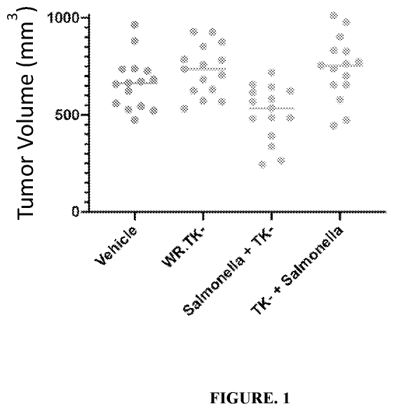

As shown in FIG. 1, the therapeutic activity of the two prime boost regimens were evaluated as compared to the control and one single agent administration. The first group Balb/c mice was a control group. The second group of Balb/c mice was injected with a single agent vaccinia virus (WR.TK−) on day 1. The third group of mice was injected with auxotrophic (aroA mutant) Salmonella Typhimurium strains (prime) on day 1 and vaccinia virus (WR.TK−) (boost) was administered on day 7. The fourth group of mice was injected with vaccinia virus (WR.TK−) (prime) on day 1 and auxotrophic (aroA mutant) Salmonella Typhimurium strains (boost) was administered on day 7. The tumor volume was then evaluated for each group on day 14 post treatment and results are shown in FIG. 1.

The results shown in FIG. 1 show enhanced therapeutic activity in the third group where salmonella was given as prime and vaccinia was given as boost. The therapeutic activity was however reduced when same recombinant microorganisms were given in opposite order (as shown for group four). Hence, these results indicate that the enhanced therapeutic activity was not purely due to an additive effect.

### Example 2: Heterologous Prime-Boost Regimen of Vaccinia Virus or Herpes Simplex Virus (HSV) or Vesicular Stomatitis Virus (VSV) in a Mouse RENCA Tumor Model

The therapeutic effectiveness of heterologous prime boost regimens in comparison to the single agent administration was tested. BALB/c mice bearing RENCA tumors (50-100 mm³) were used for each group.

Mice were dosed intratumorally with tumor selective strains of vaccinia virus with the TK deletion (labelled WO00001), the Herpes simplex virus strains with deletion of gamma 34.5 and ICP6 genes, Vesicular stomatitis virus (VSV) MΔ51 strains, or control (VFB). Mice were assigned to one of the six groups shown in Table 2.

TABLE 2
Treatment groups of RENCA-tumor mice dosed with vaccinia virus,
herpes simplex virus (HSV), or Vesicular stomatitis virus (VSV)

| Group | Group name | Prime-Boost Regimen |
|---|---|---|
| 1 | Control (VFB) | — |
| 2 | WO0001 | Vaccinia (day 1) |
| 3 | HSV + WO0001 | Vaccinia (day 1); HSV (day 7) |
| 4 | VSV + WO0001 | Vaccinia (day 1); VSV (day 7) |
| 5 | HSV only | HSV (day 1) |
| 6 | VSV only | VSV (day 1) |

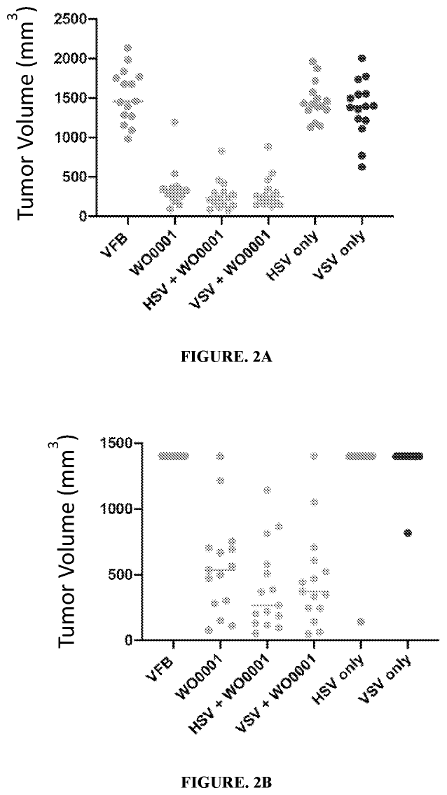

As shown in FIG. 2A and FIG. 2B, the therapeutic activity of the two heterologous prime boost combinations were evaluated as compared to a control and three single agent administrations. The first group of Balb/c mice served as a control. The second group of Balb/c mice was injected with a single agent vaccinia virus (WR.TK−) at a dose of about 1×10⁷ PFU/mouse on day 1. The third group of Balb/c mice was injected with vaccinia virus (WR.TK−) strain at a dose of about 1×10⁷ PFU/mouse on day 1 and Herpes simplex virus (gamma 34.5 and ICP6 deletion) strains at a dose of about 3×10⁶ PFU/mouse on day 7. The fourth group of Balb/c mice was injected with vaccinia virus (WR.TK−) strain at a dose of about 1×10⁷ PFU/mouse on day 1 and Vesicular stomatitis virus (MΔ51) strains at a dose of about 1×10⁷ PFU/mouse on day 7. The fifth group was injected with a single agent Herpes simplex virus (gamma 34.5 and ICP6 deletion) strains at a dose of about 3×10⁶ PFU/mouse on day 1. The sixth group of Balb/c mice was injected with a single agent Vesicular stomatitis virus (MΔ51) strains at a dose of about 1×10⁷ PFU/mouse on day 1. The tumor volume was then evaluated for each group on day 18 post treatment and results are shown in FIG. 2A and on day 21 post treatment and results are shown in FIG. 2B (N.B. at day 21, mice with tumor burden >1400 mm³ or that had been previously sacrificed due to a tumor burden >1400 mm³ were recorded as tumor burden of 1400 mm³).

The results shown in Figure. 2A and Figure. 2B demonstrate that the synergistic therapeutic activity was only seen in prime boost regimens of groups three and four where vaccinia was given as prime and HSV or VSV strains were given as boost.

TABLE 3
Sequence

| | |
|---|---|
| SEQ ID NO: 1 | AACATTCTTTGTGGATTGTTTGACAGTAGACAGTCATTTGTATACGTTAACGAATT |
| U21221.1 | TTGAAAAATTGAATCATTATTTAATCATAATATTCATTGCGCACGCGCAACACAAA |
| Staphylococcus aureus | AAGGAGACATGTCAATGACATATAGAATAAAGAAATGGCAAAAATTATCCACCATT |
| hyaluronate lyase | ACGTTATTAATGGCTGGTGTGATTACTTTGAATGGTGGTGAATTCAGAAGTGTTGA |
| (hysA) gene, complete | TAAACATCAAATCGCTGTGGCTGATACGAATGTTCAAACGCCAGATTATGAAAAAT |
| cds | TGAGGAACACATGGCTGGACGTTAACTATGGTTATGATAAGTATGATGAGAATAAT |
| | CCAGATATGAAGAAGAAGTTTGATGCTACAGAGAAAGAGGCGACGAATTTACTCAA |
| | GGAAATGAAAACTGAAAGTGGTAGGAAATACTTGTGGTCAGGAGCGGAAACCCTTG |
| | AAACTAATTCTTCTCATATGACTCGTACCTATCGTAATATTGAGAAAATCGCAGAA |
| | GCGATGAGGAATCCTAAAACCACTTTAAATACTGACGAAAATAAGAAGAAAGTGAA |
| | AGATGCCCTTGAGTGGCTGCATAAAAATGCATATGGAAAAGAACCAGATAAAAAAG |
| | TAAAAGAATTAAGTGAGAATTTTACTAAAACAACTGGCAAGAATACCAACTTAAAT |
| | TGGTGGGATTATGAAATTGGAACACCTAAATCATTAACAAATACGCTTATATTGCT |
| | GAATGATCAATTTTCAAATGAAGAAAAGAAAAAATTCACTGCCCCTATTAAAACTT |
| | TCGCCCCAGATAGTGACAAAATATTATCTTCTGTAGGAAAAGCTGAACTTGCTAAA |
| | GGCGGAAATCTAGTAGACATTTCTAAGGTGAAACTTTTAGAATGTATTATCGAAGA |
| | AGATAAAGATATGATGAAAAAGTCTATAGATTCATTTAATAAAGTCTTCACTTACG |
| | TTCAAGATTCTGCCACTGGTAAAGAACGTAATGGATTTTATAAAGATGGCTCTTAC |
| | ATTGATCATCAAGACGTTCCATACACTGGTGCTTATGGCGTTGTACTCTTAGAGGG |
| | GATTTCTCAAATGATGCCGATGATAAAAGAAACACCTTTTAATGATAAAACCCAAA |
| | ATGATACAACCTTAAAGTCATGGATTGACGACGGATTTATGCCACTCATTTATAAA |
| | GGTGAAATGATGGATTTATCACGAGGTAGAGCTATCAGTCGTGAAAATGAAACCAG |
| | TCACTCAGCATCTGCAACAGTAATGAAATCATTGTTGAGATTAAGTGATGCCATGG |
| | ATGATTCAACAAAAGCTAAGTATAAAAAGATTGTCAAATCTTCAGTAGAGTCAGAT |
| | TCAAGTTATAAACAAAATGATTATTTAAATTCATATTCGGACATAGATAAAATGAA |
| | GTCTTTAATGACAGATAACAGTATTTCTAAAAACGGATTAACACAACAACTTAAAA |
| | TATATAATGACATGGATCGTGTCACCTATCATAACAAAGACTTAGACTTTGCATTT |
| | GGTTTAAGTATGACGTCGAAAAACGTAGCACGCTATGAAAGTATCAACGGAGAGAA |
| | TTTAAAAGGTTGGCACACTGGTGCTGGAATGTCTTATTTATATAACAGCGATGTCA |
| | AACACTATCATGATAACTTCTGGGTGACAGCCGATATGAAACGTTTATCAGGTACA |
| | ACAACTTTAGACAATGAAATATTAAAAGATACGGATGATAAAAAGTCGAGTAAAAC |
| | TTTTGTTGGCGGAACAAAAGTTGATGACCAACATGCTAGTATCGGAATGGATTTTG |
| | AAAATCAGGACAAAACTTTAACTGCCAAAAAATCATATTTCATATTAAACGATAAA |
| | ATTGTCTTCTTAGGAACTGGCATTAAAAGTACTGATTCATCAAAGAATCCAGTTAC |
| | AACGATTGAAAATCGCAAAGCGAATGGGTATACGTTATATACAGACGATAAACAAA |
| | CAACCAATTCTGATAATCAGGAAAACAATTCAGTCTTTTTAGAGTCCACAGATACC |
| | AAAAAGAACATCGGTTATCATTTTTTAAACAAACCGAAAATAACTGTAAAAAAAGA |
| | AAGTCATACTGGTAAGTGGAAAGAAATAAATAAAAGTCAAAAGGATACACAAAAAA |
| | CTGATGAGTATTATGAAGTAACTCAAAAGCATTCTAATTCTGACAATAAATATGGA |
| | TATGTGTTGTATCCAGGCTTATCTAAAGATGTCTTTAAGACAAAAAAAGATGAAGT |
| | AACTGTCGTTAAGCAAGAAGATGACTTCCACGTTGTGAAAGATAATGAATCGGTTT |
| | GGGCTGGTGTTAATTATAGTAATAGCACTCAAACTTTTGACATTAACAACACTAAA |
| | GTCGAAGTTAAAGCCAAAGGTATGTTTATACTTAAAAAGAAAGATGATAACACTTA |
| | TGAATGTAGCTTCTATAATCCTGAATCTACAAATTCCGCTTCAGATATTGAATCTA |
| | AAATTTCAATGACCGGTTACTCTATTACAAACAAAAATACGTCGACTTCTAATGAA |
| | TCCGGCGTGCACTTTGAATTAACTAAATAAGCAAGAAAATCTCGACACTATACCAT |
| | TTTTATAGTGTCGAGATTTTATAAGTATCGCCATCATTCAGCTTGTAATCCGCAGA |
| | CCACATCC |
| SEQ ID NO: 2 | MTYRIKKWQKLSTITLLMAGVITLNGGEFRSVDKHQIAVADTNVQTPDYEKLRNTW |
| sp\|Q59801\|HYSA_STAA8 | LDVNYGYDKYDENNPDMKKKFDATEKEATNLLKEMKTESGRKYLWSGAETLETNSS |
| Hyaluronate lyase | HMTRTYRNIEKIAEAMRNPKTTLNTDENKKKVKDALEWLHKNAYGKEPDKKVKELS |
| OS = Staphylococcus | ENFTKTTGKNTNLNWWDYEIGTPKSLTNTLILLNDQFSNEEKKKFTAPIKTFAPDS |
| aureus (strain NCTC | DKILSSVGKAELAKGGNLVDISKVKLLECIIEEDKDMMKKSIDSENKVETYVQDSA |
| 8325) OX = 93061 | TGKERNGFYKDGSYIDHQDVPYTGAYGVVLLEGISQMMPMIKETPFNDKTQNDTTL |
| GN = hysA PE = 3 SV = 1 | KSWIDDGEMPLIYKGEMMDLSRGRAISRENETSHSASATVMKSLLRLSDAMDDSTK |
| | AKYKKIVKSSVESDSSYKQNDYLNSYSDIDKMKSLMTDNSISKNGLTQQLKIYNDM |
| | DRVTYHNKDLDFAFGLSMTSKNVARYESINGENLKGWHTGAGMSYLYNSDVKHYHD |
| | NFWVTADMKRLSGTTTLDNEILKDTDDKKSSKTFVGGTKVDDQHASIGMDFENQDK |
| | TLTAKKSYFILNDKIVFLGTGIKSTDSSKNPVTTIENRKANGYTLYTDDKQTTNSD |
| | NQENNSVFLESTDTKKNIGYHFLNKPKITVKKESHTGKWKEINKSQKDTQKTDEYY |
| | EVTQKHSNSDNKYGYVLYPGLSKDVFKTKKDEVTVVKQEDDFHVVKDNESVWAGVN |
| | YSNSTQTEDINNTKVEVKAKGMFILKKKDDNTYECSFYNPESTNSASDIESKISMT |
| | GYSITNKNTSTSNESGVHFELTK |

1. A method of treating a tumor in a subject, comprising:
administering to the subject a first recombinant microorganism,
wherein the first recombinant microorganism replicates in a tumor cell and does not replicate in a non-tumor cell or displays attenuated replication in a non-tumor cell, and
wherein the first recombinant microorganism is a virus or a bacterium;
administering to the subject a second recombinant microorganism,
wherein the second recombinant microorganism replicates in a tumor cell and does not replicate in a non-tumor cell or displays attenuated replication in a non-tumor cell, and
wherein the second recombinant microorganism is a virus; and
enhancing or eliciting an immune response to a protein expressed by a tumor associated cell that is not coded for or expressed by the first recombinant microorganism and the second recombinant microorganism.

2. The method of claim 1, wherein the immune response is demonstrated by one or more of a decrease in the volume of the tumor in the subject, a decrease in the level of expression of one or more tumor proteins in the subject or a sample from the subject, a decrease in the number of tumor sites in the subject, a change in viral load in the subject or the sample from the subject, a change in population of immune cells in the subject or the sample from the subject, a change in expression levels of an immune cell marker in the subject or the sample from the subject, an enhancement of B-cell proliferation in the subject or the sample from the subject, an enhancement of CD4+ T cell proliferation in the subject or the sample from the subject, an enhancement of CD8+ T cells proliferation in the subject or the sample from the subject, an enhancement of cytokine production in the subject or the sample from the subject, an enhancement of antigen presenting cell proliferation in the subject or the sample from the subject, or any combinations thereof.

3. The method of claim 2, wherein the immune response is demonstrated by the decrease in the level of expression of one or more tumor proteins, the change in population of immune cells, the change in expression levels of an immune cell marker, the enhancement of B-cell proliferation, the enhancement of CD4+ T cell proliferation, the enhancement of CD8+ T cells proliferation, the enhancement of cytokine production, the enhancement of antigen presenting cell proliferation, or any combinations thereof, in the subject or the sample from the subject, wherein the sample from the subject is a blood, tissue, urine, or saliva sample.

4. The method of claim 1, wherein the immune response can be detected at a time point at or after the administration of the first or second recombinant microorganism.

5. The method of claim 1, wherein the first recombinant microorganism does not replicate in the non-tumor cell.

6. The method of claim 1, wherein the first recombinant microorganism displays attenuated replication in the non-tumor cell.

7. (canceled)

8. (canceled)

9. The method of claim 1, wherein the second recombinant microorganism does not replicate in the non-tumor cell.

10. The method of claim 1, wherein the second recombinant microorganism displays attenuated replication in the non-tumor cell.

11. (canceled)

12. (canceled)

13. (canceled)

14. (canceled)

15. The method of claim 1, wherein the first recombinant microorganism is a bacterium.

16. The method of claim 15, wherein the first recombinant microorganism is an enterobacterium, a listeriaceae bacterium, or a streptococcaceae bacterium.

17-33. (canceled)

34. The method of any one of claim 1, wherein the first recombinant microorganism is a virus.

35. The method of claim 15, wherein the first recombinant microorganism is of family viridae.

36. (canceled)

37. (canceled)

38. The method of claim 1, wherein the first recombinant microorganism comprises a poxvirus, a picornavirus, an adenovirus, a parvovirus, a herpesvirus, a reovirus, a paramyxovirus, a rhabdovirus, an orthomyxovirus, or a coxsackievirus.

39-100. (canceled)

101. The method of claim 1, wherein the second recombinant microorganism is of family viridae.

102. (canceled)

103. (canceled)

104. The method of claim 101, wherein the second recombinant microorganism is a poxvirus, a picornavirus, an adenovirus, a parvovirus, a herpesvirus, a reovirus, a paramyxovirus, a rhabdovirus, an orthomyxovirus, or a coxsackievirus.

105-153. (canceled)

154. The method of claim 1, wherein the first recombinant microorganism and the second recombinant microorganism are administered to the subject simultaneously.

155. The method of claim 154, wherein the first recombinant microorganism is formulated in a delayed release composition, a sustained release composition, an immediate release composition, a stealth release composition, or any combinations thereof.

156. (canceled)

157. The method of claim 1, wherein the second recombinant microorganism is administered to the subject after the first recombinant microorganism is administered to the subject.

158. The method of claim 157, wherein the second recombinant microorganism is administered from about 1-60 days, from 1-45 days, from 1-30 days, from 1-15 days, from 1-10 days, or from 1-7 days after administration of the first recombinant microorganism.

159. The method of claim 157, wherein the second recombinant microorganism is administered about 1, 2, 3, 4, 5, 6, 7, 8, 9, 10, 11, 12, 14, 21, 28, 35, 56, or 60 days after administration of the first recombinant microorganism.

160. The method of claim 157, wherein the second recombinant microorganism is administered to the subject one, two, three, three, four, five or more times.

161. The method of claim 157, wherein the second recombinant microorganism is administered to the subject two, three, three, four, five or more times with about 1-60 days, 1-45 days, 1-30 days, 1-15 days, 1-10 days, 1-7, 1-5 days, or 1-3 days between each administration.

162-166. (canceled)

167. The method of claim 1, wherein the first recombinant microorganism is administered intra-tumorally, intradermally, subcutaneously, intraperitoneally, intramuscularly or intravenously; and the second recombinant microorganism is administered intra-tumorally, intradermally, subcutaneously, intraperitoneally, intrathecally, intramuscularly or intravenously.

168. (canceled)

169. The method of claim 1, further comprising administering an anti-cancer therapy.

170-183. (canceled)

184. A method of treatment, comprising:
administering to a subject a first recombinant microorganism,
wherein the first recombinant microorganism comprises an exogenous nucleic acid encoding a cytokine receptor,
wherein the first recombinant microorganism replicates in a tumor microenvironment, and
wherein the first recombinant microorganism is a virus or a bacterium;
administering to the subject a second recombinant microorganism,
wherein the second recombinant microorganism comprises an exogenous nucleic acid encoding a cytokine receptor,
wherein the second recombinant microorganism replicates in a tumor microenvironment, and
wherein the second recombinant microorganism is an oncolytic virus; and
enhancing or eliciting an immune response to a protein expressed by a non-tumorous cell present within the tumor microenvironment that is not coded for or expressed by the first recombinant microorganism and the second recombinant microorganism.
